SEQUENCE LISTING (2 sequences)

SEQ ID NO: 1 - DNA, 2062 bp, Human
accagtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagtgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgcgctcccgcccgcgcccagtcttctcagatgaggctgcgccctgcgggcagctctaaggaccgtcctcgcagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaag

SEQ ID NO: 2 - protein, 455 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

(19) United States
(12) Patent Application Publication
(10) Pub. No.: US 2004/0208863 A1
(43) Pub. Date: Oct. 21, 2004

(54) Anti-inflammatory activity from lactic acid bacteria

(76) Inventors: James Versalovic, Houston, TX (US); Jeremy A. Pena, Houston, TX (US); Eamonn Connolly, Lidingo (SE)

Correspondence Address: Lynn E. Barber, Post Office Box 16528, Fort Worth, TX, 76162, US

(21) Appl. No.: 10/767,317

(22) Filed: Jan. 29, 2004

Related U.S. Application Data
(60) Provisional application No. 60/443,644, filed on Jan. 30, 2003.

(51) Int. Cl. A61K 35/00; C12N 1/20

(52) U.S. Cl. 424/115; 435/252.9

(57) ABSTRACT

In the present invention, lactic acid bacteria produce soluble factors (such as peptides or proteins) that block inflammatory responses in a mechanism that depends on G proteins and is post-transcriptional to effectively block protein production or secretion by cells.

### CROSS-REFERENCE TO RELATED APPLICATIONS

This application claims priority from U.S. Provisional Application S.No. 60/443,644 filed Jan. 30, 2003, which is incorporated herein by reference.

### STATEMENT REGARDING FEDERALLY SPONSORED RESEARCH OR DEVELOPMENT

The present invention was developed in part with funds from NIH Grant No. K08-DK02705.

### FIELD OF THE INVENTION

The present invention is directed to the fields of immunology, medicine, cell biology, and molecular biology. In a specific embodiment, the present invention regards an anti-inflammatory molecule secreted from lactic acid bacteria, including Lactobacillus and other species, and methods concerning thereof.

### BACKGROUND OF THE INVENTION

Probiotics are commensal microbes with positive health benefits beyond mere nutrition (Lilly and Stillwell R. H., 1965). Commensal species of the genus Lactobacillus represent the most commonly used probiotic bacteria in clinical studies. Their ubiquitous presence and role as members of the autochthonous (indigenous) microbiota (Alvarez-Olmos and Oberhelman, 2001; Holzapfel et al., 2001; Reuter, 2001) have stimulated interest in their roles as gut-beneficial bacteria. By capsule endoscopy, Reuter (2001) describes the presence of multiple Lactobacillus species as indigenous intestinal bacteria residing in the gastrointestinal tracts of healthy children and adults. One study (Ahrne et al., 1998b) showed that Lactobacillus rhamnosus was one of the 3 most commonly found intestinal lactobacilli found in the oral and rectal mucosa of healthy human individuals. Healthy rodents including mice are also commonly colonized by lactobacilli in the stomach and intestine (Tannock, 1997). This species inhabits the oral cavity in humans and has been found in dental caries (Marchant et al., 2001). L. rhamnosus has also been found in the intestinal mucosa (Ahrne et al., 1998a) and comprises part of the vaginal flora (Pavlova et al., 2002).

Lactobacillus rhamnosus GG (LGG) was isolated from the stool of a healthy individual in 1985 by S. Gorbach and B. Goldin (Gorbach, 2000a; U.S. Pat. No. 4,839,281) and subsequent studies showed beneficial effects in patients with colitis (Gorbach et al., 1987). This organism was initially classified as Lactobacillus casei subsp. Rhamnosus, but subsequent refinements in Lactobacillus taxonomy have resulted in re-classification as L. rhamnosus (Chen et al., 2000;Mori et al., 1997). LGG colonizes the gut of rodents (Banasaz et al., 2002) and humans (Alander et al., 1997) and inhibits the growth of a variety of gram-negative and gram-positive bacteria (Dong et al., 1987). This strain has been shown to adhere to the colonic mucosa in human individuals (Alander et al., 1999) and can be recovered successfully from colonic mucosa and feces. It survives for 1-3 days in most individuals and up to 7 days in 30% of subjects. In addition to its colonization ability, the presence of LGG affects mucosal immune responses. LGG stimulates mucosal IgA responses and enhances antigen uptake in Peyer's patches (Gorbach, 2000b).

As a potential probiotic agent, multiple studies have demonstrated the ability of LGG to colonize the intestinal tract and modulate mucosal epithelial and immune responses. LGG increased enterocyte proliferation and villous size in mono-associated gnotobiotic rats (Banasaz et al., 2002). LGG also modulates the proliferation of murine lymphocyte responses ex vivo following oral administration (Kirjavainen et al., 1999) and L. paracasei alters modulatory cytokine profiles of CD4+ T lymphocytes (von der et al., 2001). In addition to adaptive immune responses, LGG has effects on innate immune responses. LGG activates nuclear factor kappa B (NF-κB) and signal transducer and activator of transcription (STAT) signaling pathways in human macrophages (Miettinen et al., 2000), and L. rhamnosus stimulates interleukin-12 (IL-12) production by macrophages (Hessle et a I., 1999). LGG also stimulates production of immunomodulatory cytokines such as IL-10 in children (Pessi et al., 2000) and may regulate pro-inflammatory responses in vivo. Effector cells of innate immunity, such as macrophages, dendritic cells and neutrophils, are the primary drivers for the majority of inflammatory responses (Janeway, Jr. and Medzhitov, 2002). The thought that innate immunity dictates the course of both innate and adaptive responses to antigens as self or non-self emphasizes the role of the innate immunity in controlling inflammation.

U.S. Pat. No. 4,314,995 regards a process concerning pharmaceutical lactobacillus preparations, particularly those having specific properties and being certain strains, the properties including growing in a culture comprising low nutrition and in a culture comprising a substance from the group of Na₂S, NH₃, lower fatty acids, or mixtures thereof. In particular embodiments the invention is directed to gastritis and enteritis.

U.S. Pat. No. 4,839,281 is directed to a particular Lactobacillus strain having ATCC Accession No. 53103 and methods related thereto, the strain being Lactobacillus rhamnosus GG.

U.S. Pat. No. 6,132,710 describes particular L. salivarius and L. plantarum strains useful for preventing neonatal necrotizing enterocolitis gastrointestinal tissue injury.

U.S. patent application Ser. No. 20020019043 A1 relates to treating inflammatory bowel disease by administering a cytokine-producing Gram-positive bacteria or a cytokine antagonist-producing Gram-positive bacterial strain. In specific embodiments, the cytokine or cytokine antagonist s elected from IL-10, a soluble TNF receptor or another TNF antagonist, an IL-12 antagonist, an interferon-y antagonist, an IL-1 antagonist, and others. In specific embodiments, the Gram-positive bacteria is genetically engineered to produce a cytokine, cytokine antagonist, and so forth.

Borruel et al. (2002) describe downregulation of TNF-α upon providing several Lactobacillus species, although the effect was not prevented by protease inhibitors.

In addition to Lactobacillus species, other lactic acid bacteria have been used as probiotic bacteria, such as Bifidobacteriium, which is used, for example, to ferment dairy product and treat intestinal infections and diarrhea, and Streptococcus (e.g., Streptococcus thermophilus) used in the food industry, and to treat diarrhea as well as intestinal and vaginal infections, and improve the nutritional value of foods by making micronutrients available to the host.

Although many different lactic acid are known to produce various factors that have antibacterial, immunomodulating and/or anti-inflammatory effects, these factors are generally complex and/or large-molecular weight products (for example, the 20 kDA protein and “additional factor(s)” of Panigrahi (International Publication No. WO 01/10448 published 15 Feb. 2001).

The present invention, however, addresses the need in the art for providing an effective contact-independent means for administering an anti-inflammatory soluble Lactobacillus or other lactic acid bacterial agent, particularly in a mechanism that comprises posttranscriptional inhibition of TNF-α and G proteins.

### BRIEF SUMMARY OF THE INVENTION

The present invention is directed to a system, methods, and compositions that are useful for the inhibition of inflammation. In some embodiments, the present invention concerns a process of isolating novel anti-inflammatory compounds from bacteria that inhibit the production of proteins (cytokines) that promote or regulate inflammation in mammals. In specific embodiments of the present invention, the inventors characterize the ability for Lactobacillus, such as LGG, or other lactic acid bacteria, to specifically inhibit pro-inflammatory cytokine production by the innate immune system. With a murine macrophage model, the present inventors demonstrate that LGG specifically inhibits TNF-α production independent of apoptosis or cytotoxic effects. LGG secretes soluble factors including proteins that diminish TNF-α production by lipopolysaccharide (LPS)- or lipoteichoic acid (LTA)-activated macrophages independent of effects on other cytokines. Furthermore, the TNF-α-inhibitory effects of LGG also antagonize stimulatory effects of Helicobacter pylori- or Helicobacter hepaticus-conditioned media.

Generally, the invention pertains directly to Lactobacillus organisms (any species of this genus) and other lactic acid bacteria, and the soluble factors that they produce and secrete into their environment. These factors (heretofore not identified specifically) inhibit cytokine (e.g. TNF-α) production following mRNA synthesis (post-transcriptional) by a G protein-dependent (G protein-coupled receptors) mechanism. Some embodiments of the present invention comprise applications in therapeutics (anti-inflammatory action) regarding the fact that lactobacilli are producing soluble factors (peptides, proteins, etc.) that block inflammatory responses in a mechanism that depends on G proteins and works at a step following mRNA synthesis to effectively block protein production or secretion by cells.

Thus, the present inventors have identified Lactobacillus and other lactic acid bacterial strains that diminish pro-inflammatory cytokine (e.g. TNF-α) and/or chemokine (e.g. IL-8) production. Soluble factors derived from bacteria in preferred embodiments inhibit expression of pro-inflammatory cytokines and result in a net anti-inflammatory effect on the immune system. Other embodiments include the bacterial organisms and soluble factors produced by these organisms.

Studies in vitro have utilized cultured macrophages (immune cells) and epithelial cells that have been activated by different stimulators. In the present invention, lactobacilli inhibit pro-inflammatory cytokine expression in a contact-independent manner, by the secretion of soluble peptide factors that bind to receptors on cells of the innate immune system and regulate cytokine expression and the linkages between innate and adaptive immunity. That is, the production of cytokines is inhibited and, in specific embodiments, that diminishes T cell (adaptive) responses. The present invention also indicates that these soluble factors inhibit and/or antagonize the pro-inflammatory effects of other pathogens.

Thus, in further specific embodiments of the present invention, Lactobacillus rhamnosus GG decreases TNF-α production in lipopolysaccharide-activated murine macrophages by a contact-independent mechanism, and L. rhamnosus GG specifically inhibits LPS- and LTA-mediated TNF-α production by primary peritoneal (in 129 SvEv) and transformed (RAW 264.7) macrophages.

In some embodiments particular Lactobacillus or other lactic acid bacterial species are preferable to others, and a skilled artisan knows how to determine optimal or preferred species from teachings provided herein. In some embodiments, specific immune effects may be species- or strain-specific. In preferred embodiments of the present invention, an effect of an anti-inflammatory secreted lactic acid bacterial compound is serum- and/or contact-independent, requiring the presence of soluble LGG immunomodulins for optimum modulatory activity.

In specific embodiments, LGG utilizes inhibitory heterotrimeric G (Gi) proteins in order to inhibit TNF-α production by macrophages. A skilled artisan recognizes that the net effect of LGG is immunomodulatory in nature, as TNF-α production is abolished while IL-10 is unaffected. In specific embodiments, intestinal Lactobacilli produce soluble protein factors that presumably bind to cell surface receptors and inhibit synthesis or secretion of TNF-α, independent of pro-apoptotic effects or cell necrosis.

In specific embodiments of the present invention, a compound of the present invention is at least one soluble agent from Lactobacillus or other lactic acid bacterial culture, wherein the agent comprises anti-inflammatory activity, anti-cytokine production activity, G protein receptor binding activity, or a combination thereof. In specific embodiments, the compound is a polypeptide, such as a protein or peptide, or a non-polypeptide, such as a nucleic acid molecule or a small molecule. As used herein, a “polypeptide” is a molecular chain of at least two amino acids, and includes small peptides. In the preferred embodiments, the compound is a small peptide as determined in the experiments discussed herein.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 provides a schematic of a LGG-macrophage bioassay. Macrophages are stimulated with purified LPS from E. coli. Activation is characterized by morphologic changes, such as vacuolization and extrusion of cellular processes. Additionally, activation also results in secretion of pro-inflammatory cytokines, such as TNF-α. The presence of putative immunomodulins made by laciobacilli, may block LPS-mediated production of TNF-α.

FIGS. 2A and 2B show that LGG-conditioned media inhibits TNF-α production by LPS-activated macrophages. In contrast to E. coli, media conditioned by Lactobacillus or other lactic acid bacterial species do not induce production of TNF-α in RAW 264.7 macrophages as measured by quantitative ELISA (FIG. 1A). Only conditioned media from LGG inhibited LPS-mediated TNF-α production by macrophages (B). Conditioned media of selected bacterial species are represented: Lacid 4796 (L. acidophilus ATCC 4796), LGG (L. rhamnosus GG), Lreut 55148 (L. reuteri ATCC 55148), Ec Nissle (E. coli Nissle). Media controls: MRS (deMan, Rogosa, Sharpe) and LB (Luria-Bertani).

FIG. 3 demonstrates that inhibition of TNF-α production by LGG is reversible. RAW 264.7 macrophages were activated with LGG-cm+LPS or LPS alone. Five hours post-activation, cell culture medium was assayed for TNF-α using quantitative E LISA. Spent culture media was removed and replenished with fresh media. Macrophages were allowed to grow overnight, then re-challenged with LPS alone. Culture media was assayed for TNF-α 5 h after LPS re-challenge (LPS Re-challenge). LPS (E. coli 0127:B8-derived lipopolysaccharide).

FIGS. 4A and 4B show that macrophage activation by LPS and immunomodulatory effect of LGG is serum-independent. RAW 264.7 macrophage bioassay was performed in FBS-free conditions to determine whether serum-soluble co-factors are required for observed effects on TNF-α production as measured by quantitative ELISA. No significant differences were noted between FBS-supplemented (FIG. 4A) and FBS-free conditions (FIG. 4B). MRS (DeMan, Rogosa, Sharpe media), LPS (E. coli 0127:B8-derived lipopolysaccharide).

FIGS. 5A through 5B demonstrate that LGG-conditioned media inhibits TNF-α production by LTA-activated macrophages. Purified LTA from three different Gram-positive bacteria were used to stimulate macrophages with or without LPS (FIG. 5A). In the presence of LGG-conditioned media, TNF-α production was diminished as measured by quantitative ELISA (FIG. 5B). Media only/MRS (DeMan, Rogosa, Sharpe media), Saur (Staphylococcus aureus), Efaec (Enterococcus faecalis) Bsub (Bacillus subtilis), LTA (lipoteichoic acid), LPS (E. coli 0127:B8-derived lipopolysaccharide).

FIG. 6 shows that TNF-α/IL-10 ratios are diminished in presence of LGG. Cytokine levels of LPS-activated macrophages were measured using mouse-specific multi-cytokine antibody-bead sandwich immunoassays in a Luminex 100 instrument. Levels of IL-10 and TNF-α in LGG-cm+LPS-stimulated macrophage were compared relative to macrophages exposed to LPS alone. LGG (L. rhamnosus-conditioned media) and LPS (E. coli 0127:B8-derived lipopolysaccharide).

FIG. 7 demonstrates that LGG-derived factors antagonize activation of macrophages by Helicobacter spp. but not by E. coli. Macrophages were activated with either LPS-supplemented Helicobacter- or E- coli-conditioned media or Gram-negative-conditioned media alone. LGG-conditioned media was added to Helicobacter- or E. coli-conditioned media (1:1 ratio) to determine if LGG could decrease TNF-α production in Helicobacter-activated macrophages using quantitative ELISA. Hp (H. pylori-conditioned media), LGG (L. rhamnosus-conditioned media), Hh (H. hepaticus-conditioned media), and Ec Nissle (E. coli Nissle-conditioned media).

FIG. 8 shows that LGG-derived proteins confer immunomodulatory effects. Macrophages were activated with a mixture of LPS and modified LGG-conditioned media and TNF-α production measured by quantitative ELISA. Conditioned media was subjected to different treatments prior to mixing with LPS: untreated control (unmodified), freeze-thaw cycling (F/T), heat-denaturation (heat), DNase I treatment (DNase) and Proteinase K digestion followed by heat inactivation of Proteinase K (PK).

FIG. 9 demonstrates the effect of bacteria-conditioned media on LPS-activated macrophages. Macrophages were activated with a mixture of LPS and bacteria-conditioned media. Culture media was tested 5h post-activation for TNF-α. L. acidophilus 4796 significantly increased TNF-α production compared to macrophages activated with MRS+LPS only (p<0.01) while L. reuteri ATCC 55148 had no effect. LGG significantly decreased TNF-α production (p<0.01). Gram-negative bacteria such as E. coli, significantly increased TNF-α production compared to culture media alone.

FIG. 10 demonstrates that immunomodulation is not due to pH effects. To control for lactic acid production and reduced pH effects, acidified MRS media (pH 4) was tested and did not affect TNF-α levels without the presence of LGG-cm. Conditioned media derived from other lactic acid bacteria did not inhibit TNF-α secretion and was inconsistent with general pH effects due to lactic acid production.

FIG. 11 provides effects of LGG-conditioned media on LTA-activated macrophages. Macrophages were activated with LTA derived from S. aureus, B. subtilis, and E. faecalis. LGG-conditioned media significantly decreased pro-inflammatory cytokine expression in LTA-activated macrophages compared to MRS media alone (p<0.01).

FIG. 12 shows that an immunomodulatory effect is retained in the 10 kDA fraction. LGG-conditioned media was fractionated using size exclusion filters. The media control is indicated by “mock”. Inhibition of TNF-α production was observed in the <10 kDa fraction. In contrast, the >10 kDa fraction lost immunomodulatory activity. Taken together with previous data from the inventors, this indicates that a small peptide is responsible for immunomodulation and does not require serum.

FIG. 13 shows that immunomodulation utilizes heterotrimeric G proteins. Following PTx treatment, RAW 264.7 cells were stimulated with LPS alone or co-cultured with Lactobacillus-conditioned media (CM)(medium conditioned by growth of the particular lactic acid bacterial strain(s)). The ability of Lactobacillus-conditioned media to exert TNF-inhibitory effects was partially diminished when RAW 264.7 cells were intoxicated with PTx.

FIG. 14 demonstrates that TNF-α/IL-10 ratios are diminished in presence of LGG. Cytokine levels of LPS-activated macrophages were measured using mouse-specific multi-cytokine antibody-bead sandwich immunoassays in a Luminex 100 instrument. Levels of IL-10 and TNF-α in LGG-cm+LPS-stimulated macrophage were compared relative to macrophages exposed to LPS alone. LGG (L. rhamnosus-conditioned media) and LPS (E. coli 0127:B8-derived lipopolysaccharide).

FIG. 15 illustrates a cluster diagram of Lactobacillus strains. Lactobacillus spp is indicated by “L.spp.”; Lactobacillus reuteri is indicated by “L.r.”; Lactobacillus johnsonii is indicated by “Lj.”and “Wild-type” is indicated by “W-t”. In the right-hand column, “t” refers to “top” and “b” refers to “bottom” with respect to DNA fragment location on the gel DNA profile.

FIG. 16 illustrates a cluster diagram of Lactobacillus strains. Abbreviations are as in FIG. 15.

FIG. 17 shows that TNF-α-inhibitory (“immunomodulin”) activity requires the presence of G protein Gi α 2. Resident peritoneal macrophages from Giα2-deficient mice (129 Sv background) were stimulated with LPS alone (MRS+LPS) or with LPS and Lactobacillus-derived CM (LGG+LPS, MM7+LPS, CF48+LPS). Relative TNF-α levels were determined by quantitative ELISA (Quantikine M, R&D Systems). MRS, de Man, Rogosa, Sharpe medium; LGG, L. rhamnosus strain GG; MM7, L. reuteri strain MM7; CF48, L. reuteri strain CF48. WT, wild type Giα2^{+/+} macrophages; Hetzyg, heterozygous knockout Giα2^{±} macrophages; Homzyg, homozygous knockout Giα2^{-/-} macrophages.

### DETAILED DESCRIPTION OF THE INVENTION

Definitions

The term “a” or “an” as used herein in the specification may mean one or more. As used herein in the claim(s), when used in conjunction with the word “comprising”, the words “a” or “an” may mean one or more than one. As used herein “another” may mean at least a second or more.

The term “colitis” as used herein refers to an acute or chronic inflammation of the colon, in specific embodiments the membrane lining the large bowel. Symptoms of colitis may include abdominal pain, diarrhea, rectal bleeding, painful spasms (tenesmus), lack of appetite, colonic ulcers, fever, and/or fatigue. The term “contact-independent” as used herein refers to the embodiment wherein cell:cell contact is not required. In a specific embodiment, the utilization of soluble factors circumvents the requirement for cell:cell contact.

The term “probiotic” as used herein refers to at least one organism that contributes to the health and balance of the intestinal tract. In specific embodiments, it is also referred to as “friendly”, “beneficial”, or “good” bacteria, which when ingested assists in the maintenance of a healthy intestinal tract and assists in combating illness and/or disease.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art: Except insofar as any conventional media or agent is incompatible with the vectors or cells of the present invention, its use in therapeutic compositions is contemplated. Supplementary active ingredients also can be incorporated into the compositions.

The term “therapeutically effective amount” as used herein refers to an amount that results in an improvement or remediation of the disease, disorder, or symptoms of the disease or condition.

The term “treating” and “treatment” as used herein refers to administering to a subject a therapeutically effective amount of a the composition so that the subject has an improvement in the disease. The improvement is any improvement or remediation of a symptom or symptoms. The improvement is an observable or measurable improvement. Thus, one of skill in the art realizes that a treatment may improve the disease condition, but may not be a complete cure for the disease.

The Present Invention

The invention herein comprises a compound secreted from lactic acid bacteria that comprises anti-inflammation activity. The lactic acid bacteria are preferably selected from the group consisting of Lactobacillus is L. acidophilus, L. animalis, L. rhamnosus GG, L. johnsonii, L. murinus, L. plantarum, L. reuteri, L. salivarius, L. paracasei, L. delbrueckii, L. fermentum, L. brevis, L. buchneri, L. kefi, L. casei, L. curvatus, L. coryniformis, Brevibacterium, Streptococcus thermophilus, and a mixture thereof. The compound is preferably a polypeptide that preferably comprises receptor-binding activity, as well as cytokine expression regulating activity, chemokine expression regulating activity, or both. The invention also comprises a kit that includes at least one isolated bacterium as above; an isolated bacterium that produces the compound and may be capable of secreting the compound; and a method of reducing cytokine expression in a cell, in which the cytokine may be TNF-α, the cell may be an immune cell, such as a macrophage. The invention herein further comprises a method of inhibiting inflammation in an individual, for example, as found in conditions such as colitis, arthritis, synovitis, polymyalgia rheumatica, myositis, or sepsis, comprising the step of delivering a therapeutically effective amount of lactic acid bacteria to the individual, wherein the lactic acid bacteria may inhibit the inflammation by a contact-independent mechanism. In another embodiment, the lactic acid bacteria are further defined as producing a soluble compound that binds to a receptor on an immune cell. This method may be further defined as inhibiting, at least partially, in cell cytokine production, cytokine secretion, chemokine production, or a combination thereof. The inhibiting step in another embodiment is further defined as comprising inhibiting the cytokine production, cytokine secretion, chemokine production, or a combination thereof, through inhibitory heterotrimeric G (Gi) protein activity. Preferably the cytokine is TNF-α. In another preferred embodiment the chemokine is IL-8. In additional embodiments, the lactic acid bacteria are administered in combination with at least one additional therapeutic agent, such as corticosteroids, sulphasalazine, derivatives of sulphasalazine, immunosuppressive drugs, cyclosporin A, mercaptopurine, azathioprine, and a mixture thereof.

Animal studies and human clinical trials have shown that Lactobacillus can prevent or ameliorate inflammation in chronic colitis. However, molecular mechanisms for this effect have not been clearly elucidated. The present inventors determined that lactobacilli and other lactic acid bacteria are capable of down-regulating pro-inflammatory cytokine responses induced by the enteric microbiota. The Examples provided herein address whether lactobacilli diminish production of tumor necrosis factor alpha (TNF-α) by murine macrophages and alter the TNF-α/interleukin-10 (IL-10) balance, in vitro. When media conditioned by Lactobacillus rhamnosus GG (LGG) are co-incubated with lipopolysaccharide (LPS) or lipoteichoic acid (LTA), TNF-α production is significantly inhibited compared to controls, whereas IL- 10 synthesis is unaffected. Interestingly, LGG-conditioned media also decreases TNF-α production of E. coli- and Helicobacter-conditioned media-activated peritoneal macrophages. Lactobacillus species and other lactic acid bacteria may be capable of producing soluble molecules that inhibit TNF-α production in activated macrophages. Since over-production of pro-inflammatory cytokines, especially TNF-α, is implicated in pathogenesis of chronic intestinal inflammation, enteric Lactobacillus-mediated inhibition of pro-inflammatory cytokine production and alteration of cytokine profiles highlight an important immunomodulatory role for commensal bacteria in the gastrointestinal tract.

In some embodiments of the present invention, Lactobacillus and other lactic acid bacterial species have been used in probiotic strategies for gastrointestinal infections and inflammatory bowel disease. Gᵢ alpha subtype 2 (G_{αi}2)-deficient mice develop colitis that mimics the pathological lesions of ulcerative colitis in humans. The present inventors demonstrate that particular isolates of Lactobacillus are capable of decreasing lipopolysaccharide (LPS)-induced TNF-α production in both primary and transformed macrophages as a primary mechanism of probiotic action. Furthermore, in some embodiments Lactobacillus or other lactic acid bacterial species utilize inhibitory heterotrimeric G (Gᵢ) proteins in order to inhibit TNF-α production by macrophages. Resident peritoneal macrophages were recovered from G_{αi}2-deficient mice and wild-type 129Sv mice. Primary macrophages were stimulated with purified E. coli LPS alone or co-cultured with conditioned media from Lactobacillus species. RAW 264.7 gamma (NO−) macrophages were exposed to a Gᵢ protein inhibitor, pertussis toxin (PTx), in order to ablate Gᵢ protein-dependent responses. Following PTx treatment, RAW 264.7 cells were stimulated with LPS alone or co-cultured with Lactobacillus-conditioned media (CM). Levels of TNF-α, in macrophage culture supernatants, were measured by quantitative ELISA. As a model organism, Lactobacillus rhamnosus GG-CM inhibited TNF-α production in wild-type 129Sv-derived peritoneal macrophages (135 ρg/ml) and RAW 264.7 cells (150 ρg/ml) compared to primary and transformed macrophages exposed to LPS alone (1000 ρg/ml and 1500 ρg/ml, respectively). In contrast, primary macrophages from G_{αi}2-deficient mice produced high levels of TNF-α following exposure to LPS and Lactobacillus-CM. Levels of TNF-α production in cells derived from homozygous G_{αi}2 knockout mice were almost twice as much as cells from heterozygous animals (1800 versus 960, ρg/ml). The ability of Lactobacillus-CM to exert TNF-inhibitory effects was also partially diminished when RAW 264.7 cells were intoxicated with PTx (650 pg/ml). Thus, Lactobacillus and other lactic acid bacterial species isolated from humans inhibit macrophage TNF-α production by a Gᵢ protein-dependent mechanism.

A skilled artisan recognizes that in vivo models are useful in embodiments of the present invention. For example, in a specific embodiment, an in vivo model may be used to test the safety or efficacy of a compound from Lactobacillus or other lactic acid bacteria, such as secreted from Lactobacillus, that is suspected of having anti-inflammatory activity, anti-TNF-α activity, anti-chemokine activity, or a combination thereof. One example of such as model is HLA-B27 transgenic rats wherein the overexpression of the gene for the MHC class I molecule HLA-B27 leads to the development of colitis, gastroduodenitis, peripheral arthritis and spondylitis (Rath et al., and references cited therein). Other examples of models are well known in the art (Aranda et al., 1997; Cong et al., 1998; Contractor et al., 1998; Dianda et al., 1997; Garcia-Lafuente et al., 1997; Kuhn et al., 1993; Onderdondo et al., 1981; Veltkamp et al., 2001; Yamada et al., 1993). In further specific embodiments, immune cells obtained from such a model are useful, such as for assaying for changes in cytokine and/or chemokine production.

The present invention in specific embodiments regards any species of lactic acid bacteria, including any species of the genus Lactobacillus, including L. acidophilus ATCC 4796, L. animalis ATCC 35046, L. rhamnosus GG ATCC 53103, L. johnsonii ATCC 33200, L. murinus ATCC 35020, L. plantarum ATCC 14917, L. plantarum ATCC 49445, L. reuteri ATCC 53608, L. reuteri ATCC 55148, L. salivarius ATCC 11471, L. paracasei, L. delbrueckii, L. coryniformis, Bifidobacterium, Streptococcus thermophilus, or a mixture thereof. In some embodiments of the present invention, a nucleic acid sequence encoding TNF-α is utilized, such as to monitor its expression level. An example of a TNF-α sequence is comprised in SEQ ID NO:1 (GenBank Accession No. A21522). In similar embodiments, TNF-α protein levels are monitored, such as by using antibodies to at least a portion of SEQ ID NO:2 (CAA01558). A skilled artisan recognizes how to obtain other useful sequences, such as by accessing them from publicly available databases, including the National Center for Biotechnology Information's GenBank database.

Exemplary methods of isolating a particular Lactobacillus or other lactic acid bacterial strain are known in the art, such as described in U.S. Pat. No. 4,839,281, incorporated by reference herein in its entirety.

In specific embodiments of the present invention, a compound of the present invention is at least one soluble agent from Lactobacillus or other lactic acid bacteria, wherein the agent comprises anti-inflammatory activity, anti-cytokine production activity, G protein receptor binding activity, or a combination thereof. In specific embodiments, the compound is a polypeptide, such as a protein or peptide, or a non-polypeptide, such as a nucleic acid molecule or a small molecule. In a specific embodiment, the anti-inflammatory is G protein receptor ligand. A skilled artisan recognizes how to obtain and/or isolate the secreted compound by standard methods in the art. For example, the compound may be purified by testing for activity in different fractions, followed by additional fractionating and testing for activity. Activities to be tested for include protease sensitivity, anti-inflammatory activity, anti-cytokine or chemokine expression activity, G protein receptor binding activity, or a combination thereof. In specific embodiments, cytokine expression is monitored, examples of which include interleukins IL-1, IL-6, IL-12, IL-10, and/or TGF.

In specific embodiments, a 2-D gel is utilized in identifying the compound.

Lactobacillus

Lactobacilli are usually rod-shaped, varying from short bent rods to long and slender rods. Most species are homofermentative, although some are heterofermentative. Homofermentative species produce lactic acid as a major product, wherein some grow at 45° C., comprise long rods and comprise glycerol teichoic acids (such as L. delbrueckii and L. acidophilus), whereas other homofermentative species grow at 15° C., have variable growth at 45° C., are short rods and coryneforms, and comprise ribitol and glycerol teichoic acids (such as L. casei, L. plantarum, and L. curvatus). Heterofermentative species, such as L. fermentum, L. brevis, L. buchneri, and L. kefir) produce about 50% lactic acid from glucose and produce CO₂ and ethanol.

Cytokines and Chemokines

In a specific embodiment of the present invention, the production of a cytokine is inhibited upon the presence of a Lactobacillus-secreted compound. A cytokine is herein referred to as a cell-derived hormone-like polypeptide that regulates cellular replication, differentiation, and/or activation in processes concerning host defense and repair.

A n enormous number of cytokines are known in the art. Examples are illustrated in Table 1 (reproduced from a website of Dalhousie Medical School).

TABLE 1
EXEMPLARY CYTOKINES

| Cytokine | Principle Source | Principle activities |
|---|---|---|
| IL-1 | Macrophage | T, Bcellactivation; fever; in- |
| | flammation | |
| IL-2 | T cells | T cell proliferation |
| IL-3 | T cells | Growth of many cell types |
| IL-4 | T cells | B cell growth and differentiation |
| IL-5 | T cells | B cell, eosinophil growth |
| IL-6 | macrophages, T cells | B cell stimulation, inflammation |
| IL-7 | stromal cells | Early B and T cell differentia- |
| | tion | |
| IL-8 | macrophages | Neutrophil (PMN) attraction |
| IL-9 | T cells | mitogen |
| IL-10 | T cells | Inhibits Th1 cytokine production |
| IL-11 | Bone marrow stroma | Hematopoeisis |
| IL-12 | APC | Stimulates T, NK cells |
| IL-13 | T cells | Similar to IL-4 |
| IL-14 | dendritic cells, T cells | B cell memory |
| IL-15 | T cells | same as IL-2 |
| IL-16 | — | — |
| IFNα | Most cells | Anti-viral |
| IFNβ | Most cells | inflammation, activates macro- |
| | phages | |
| TGFβ | macrophages, lymphocytes | depends on target |
| TNFα | Macrophage | Inflammation; tumor killing |
| TNFβ | T cells | Inflammation; tumor killing; |
| | | enhance phagocytosis |

All cytokines have certain properties in common. They are all small molecular weight peptides or glycopeptides. Many are produced by multiple cell types such as lymphocytes, monocytes/macrophages, mast cells, eosinophils, even endothelial cells lining blood vessels. Each individual cytokine can have multiple functions depending upon the cell that produces it and the target cell(s) upon which it acts (called pleiotropism). Also, several different cytokines can have the same biologic function (called redundancy). Cytokines can exert their effect through the bloodstream on distant target cells (endocrine), on target cells adjacent to those that produce them (paracrine) or on the same cell that produces the cytokine (autocrine). Physiologically it appears that most cytokines exert their most important effects in a paracrine and/or autocrine fashion. Their major functions appear to involve host defense or maintenance and repair of the blood elements (Table 1).

A skilled artisan recognizes cytokines are categorized by their major specific function(s), and there are four major categories of cytokines: interferons, colony stimulating factors, tumor necrosis factors, and interleukins. Interferons interfere with viral replication, and there are three major types based upon the source of the interferon. Interferon alpha (IFNα) is produced by the buffy coat layer from white blood cells and is used in treatment of a variety of malignant and immune disorders. Interferon beta (IFNβ) is produced by fibroblasts and is currently being evaluated in the treatment of multiple sclerosis. Interferon gamma (IFNγ) is produced by activated T cells and is an important immunoregulatory molecule, particularly in allergic diseases.

The colony stimulating factors support the growth and differentiation of various elements of the bone marrow. Many are named by the specific element they support, such as granulocyte colony stimulating factor (G-CSF), macrophage colony stimulating factor (M-CSF), and granulocyte-macrophage colony stimulating factor (GM-CSF). Other CSFs include Interleukin (IL) -3, which can stimulate a variety of hematopoietic precursors; and c-Kit ligand (stem cell factor).

The tumor necrosis factors (TNF) cause a hemorrhagic necrosis of their tumor upon injection. TNFα is produced by activated macrophages and TNFβ is produced by activated T cells (both TH and CTL). Attempts have also been made to use the TNFs clinically to treat human tumors, but due to their extremely narrow therapeutic window (efficacy vs. toxicity), few view this as a useful stand-alone cancer therapy.

The largest group is the interleukins, so named because their fundamental function appears to be communication between (inter-) various populations of white blood cells (leucocytes-leukin). Interleukins (IL) are given numbers. They are produced by a variety of cell types such as monocytes/macrophages, T cells, B cells and even non-leucocytes.

Chemokines are a family of structurally related glycoproteins that comprise effective leukocyte activation and/or chemotactic activity. They are 70 to 90 amino acids in length and approximately 8 to 10 kDa in molecular weight. Most of them fit into two subfamilies having four cysteine residues, dependent on whether the two amino terminal cysteine residues are immediately adjacent or separated by one amino acid. The α chemokines, also known as CXC chemokines, comprise a single amino acid between the first and second cysteine residues; the β, or CC, chemokines have adjacent cysteine residues. Most CXC chemokines are chemoattractants for neutrophils, whereas CC chemokines generally attract monocytes, lymphocytes, basophils, and eosinophils.

Two additional small sub-groups of chemokines are known. The C group has one member (lymphotactin). It lacks one of the cysteines in the four-cysteine motif, but shares homology at its carboxyl terminus with the C-C chemokines. The C chemokine seems to be lymphocyte specific. The fourth subgroup is the C-X3-C subgroup. The C-X3-C chemokine (fractalkine/neurotactin) has three amino acid residues between the first two cysteine. It is tethered directly to the cell membrane via a long mucin stalk and induces both adhesion and migration of leukocytes.

G Protein Receptors

In a preferred embodiment of the present invention, a secreted Lactobacillus compound causes indirectly or directly action on a G protein receptor in a cell. A skilled artisan recognizes that a G protein normally lies near the receptor in an inactive, quiet state. When the receptor gets activated by ligand binding, it will rapidly trigger the G protein. The G protein responds by switching itself on into an active state. Once in the active state, the G protein will send signals further into the cell, one signal being, either directly or indirectly, reduction in cytokine and/or chemokine expression (such as posttranscriptionally). However, the G protein will remain in the active state for only a brief period of time, after which it will shut itself off. In effect, the G protein acts like a binary switch that, once turned on, will remain on for a limited period of time before it shuts itself off.

The G protein's two states (on or off) are determined by the guanine nucleotide that it binds (hence the term G protein). When it is inactive it binds GDP, but when it is active it binds GTP. Accordingly, the resting state off form of the G protein comprises bound GDP. When a ligand-activated receptor triggers it, the G protein releases its bound GDP and allows a GTP molecule to bind, and this GTP-bound form of the G protein represents the active ON configuration of the G protein. While in the activated state, the G protein effects downstream signals. After a short period of time (seconds or less), the G protein will then hydrolyze its own GTP down to GDP, thereby shutting itself off. This hydrolysis represents a negative feedback mechanism, which ensures that the G protein is only in the active, signal-emitting on mode for a brief period of time.

Examples of G protein receptors in immune cells are well known in the art, but specific examples include at least CCR1; CCR4; CXCR1; CXCR2; CXCR4; HM63; FPR1; EX33; the EGF-TM7 group, which comprises mouse F4/80, human EGF module-containing mucin-like hormone receptor (EMR) 1, human EMR2, and human and mouse CD97. A skilled artisan is aware of a variety of sources listing G protein receptors, including databases available on the World Wide Web, such as the G Protein Coupled Receptor Database.

Posttranscriptional Modification

In one embodiment of the present invention, cytokine production (also referred to as expression) and/or chemokine production is altered in response to providing a Lactobacillus-produced soluble molecule. A skilled artisan recognizes that this post-transcriptional modification preferable reduces cytokine production, thereby providing anti-inflammatory effects. In specific embodiments, post-transcriptional modification of more than one cytokine and/or chemokine occurs.

Specific examples of post-transcriptional modification are well known in the art. Examples include at least: utilization of multigenic transcription units; utilization of alternative promoters; alternative splicing; alternative polyadenylation; post-translational cleavage; posttranscriptional silencing, such as induced by double-stranded RNA (dsRNA) (known as RNA interference (RNAi)); C to U RNA editing; phosphorylation; antisense transcription from a bidirectional promoter; La protein binding (La protects RNAs from 3′ exonucleolytic digestion and also contributes to their nuclear retention); Q/R RNA editing; base deamination RNA editing; G-to-A editing; C-to-U editing; degradation; or a combination thereof.

As indicated, RNA editing is one form of posttranscriptional modification. RNA editing results in the generation of nucleotides within an RNA transcript that do not match the bases present within the genome. Mammalian RNA editing events, usually cytidine-to-uridine and adenosine-to-inosine conversions, are predominantly mediated by base deamination.

A skilled artisan is aware of examples of factors mediating the stability of TNFα, such as in myeloid cells stimulated with lipopolysaccharide (Mahtani et al., 2001, and references cited therein). Specifically, Mahtani and coworkers show that phosphorylation of tristetraprolin is mediated by the p38-regulated kinase MAPKAPK2, providing a direct link to mechanisms that regulate TNFα gene expression at a posttranscriptional level.

Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs that are more active or stable than the natural molecules, which have different susceptibility to alteration, or which may affect the function of various other molecules. In one approach, one would generate a three-dimensional structure for a polypeptide secreted from Lactobacillus or other lactic acid bacteria, particularly comprising anti-inflammatory activity, or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate a specific antibody to the polypeptide secreted from Lactobacillus or other lactic acid bacterial species, particularly comprising anti-inflammatory activity, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved anti-inflammatory activity or which act as stimulators, inhibitors, agonists, or antagonists of a cytokine or a chemokine, or molecules affected by function of a cytokine or chemokine. Sufficient amounts of a compound of the present invention can be produced to perform crystallographic studies. In addition, knowledge of the polypeptide sequences permits computer-employed predictions of structure-function relationships.

The present invention also encompasses the use of various animal models. By developing or isolating mutant cell lines that comprise increased levels of TNF-α, one can, in some embodiments, generate colitis models in rodents, such as mice, that will be highly predictive of same in humans and other mammals. Transgenic animals that lack a wild-type cytokine and/or chemokine may be utilized as models for colitis development and treatment.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intraenteral injection.

Determining the effectiveness of a compound in vivo may involve a variety of different criteria. Such criteria include, but are not limited to, survival, reduction of colitis symptom or symptoms, inhibition or prevention of colitis, increased activity level, and improved colonic function.

Pharmaceutical Compositions and Routes of Administration

Compositions of the present invention may have an effective amount of a Lactobacillus-secreted anti-inflammatory compound for therapeutic administration for a colon disease, joint disease, or any inflammatory condition, such as a systemic inflammatory condition, and, in some embodiments, in combination with an effective amount of a compound (second agent) that is an anti-colitis disease agent and/or anti-inflammation agent. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

Delivery vehicles, vectors, and/or pharmaceutical compositions of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for prevention and/or treatment of an inflammatory disease, such as colitis, may be assembled together in a kit. When the components of the kit are provided in one or more liquid solutions, the liquid solution preferably is an aqueous solution, with a sterile aqueous solution being particularly preferred.

For in vivo use, an anti-inflammation disease agent and/or anti-colitis agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the anti-colitis disease drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye-dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of intestinal tissues may be accomplished in any one of a variety of ways. Plasmid vectors and retroviral vectors, adenovirus vectors, and other viral vectors all present means by which to target intestinal tissue. The inventors anticipate particular success for the use of liposomes to target the polypeptide of the present invention or a nucleic acid encoding same to cells, examples of which include immune cells. For example, DNA encoding the polypeptide may be complexed with liposomes in the manner described above, and this DNA/liposome complex is injected into patients with inflammatory disease, wherein intravenous injection can be used to direct the DNA to any cell. Directly injecting the liposome complex into the proximity of the diseased tissue can also provide for targeting of the complex with some forms of inflammatory disease. Of course, the potential for liposomes that are selectively taken up by a population of cells exists, such as immune cells, and such liposomes will also be useful for targeting the gene.

Those of skill in the art will recognize that the best treatment regimens for using a compound of the present invention to treat intestinal tissue can be straightforwardly determined. This is not a question of experimentation, but rather one of optimization, which is routinely conducted in the medical arts. In one exemplary embodiment, in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of compound used in mice. In certain embodiments it is envisioned that the dosage may vary from between about 1 mg polypeptide-encoding DNA/Kg body weight to about 5000 mg polypeptide-encoding DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg polypeptide-encoding DNA/Kg body to about 20 mg polypeptide-encoding DNA/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In a specific embodiment of the present invention, the compound is administered by mouth as pill or capsule, or, in an alternative embodiment, by rectum. For rectum administration, assistance by a device, such as an endoscope and/or a colonoscope, may be useful.

Kits

Any of the compositions described herein may be comprised in a kit. In a non-limiting example, the stem cells, lipid, and/or additional agent, may be comprised in a kit. The kits will thus comprise, in suitable container means, the stem cells and a lipid, and/or an additional agent of the present invention.

The kits may comprise a suitably aliquoted stem cells, lipid and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the stem cells or the pharmacological composition of the present invention, lipid, additional agent, and any other reagent containers in close confinement for commercial sale. Such containers may include injection or blow-molded plastic containers into which the desired vials are retained.

Therapeutic kits of the present invention are kits comprising the stem cells. Such kits will generally contain, in suitable container means, a pharmaceutically acceptable formulation of the stem cells. The kit may have a single container means, and/or it may have distinct container means for each compound.

When the components of the kit are provided in one and/or more liquid solutions, the liquid solution is an aqueous solution, with a sterile aqueous solution being particularly preferred. The stem cell compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the stem cells are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate the stem cell composition within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### LGG-Mediated Inhibition of TNF-α Production by LPS-Activated Macrophages

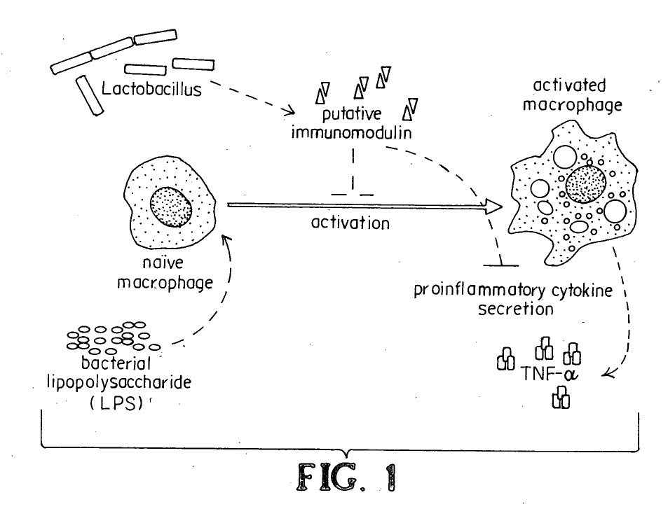

We developed an in vitro bioassay to look at the ability of lactic acid bacterial species to down-regulate inflammatory responses in cultured macrophages (FIG. 1). Cells of the innate immune system utilize germ line-encoded pattern recognition receptors (PRRs) to recognize pathogen-or commensal-associated molecular patterns (P/CAMPs). One such P/CAMP is bacterial LPS, which that serves as a ligand to the PRR, Toll-like receptor 4 (TLR4) (Lien et al., 2000;Poltorak et al., 1998). We used RAW 264.7 macrophages, a transformed peritoneal macrophage line from BALB/c mice, as reporter cells (Raschke et al., 1978). Both wild-type RAW 264.7 macrophages and a spontaneous mutant, RAW 264.7 gamma NO(−) were compared. The gamma NO(−) cell is a spontaneous mutant requiring both IFN-γ and LPS for production of nitric oxide and full activation (Lowenstein et al., 1993). Briefly, RAW 264.7 macrophages were cultured and exposed to LPS, and macrophage culture supernatants were collected at 30 min, 1, 3, 5, 7, 9, 12 and 24 h post-activation. Maximal TNF-α secretion, after LPS activation, was reached at approximately 5 hours, with no significant differences when compared to 24 h post-activation, as measured by quantitative ELISA. Levels of TNF-α production were noted to be higher in wild-type macrophages versus the gamma NO (−) cells (levels per 50,000 cells: >2500 ρg/ml and 2000-2500 ρg/ml, respectively). It must be noted that these levels may overestimate the levels of TNF-α homotrimers as quantitative ELISAs are designed to detect all forms of TNF-α including monomers and dimers.

Viable, intact ultraviolet light (UV)-killed, and sonicated Lactobacillus cells had different effects on LPS-mediated activation of macrophages in co-incubation experiments. Exposure of macrophages to either viable or UV-killed bacteria did not induce TNF-α secretion, whereas bacterial c ell sonicates elicited high levels of TNF-α (data not shown). Both intact viable and UV-killed LGG cells failed to abrogate TNF-α production when macrophages were co-exposed to LPS.

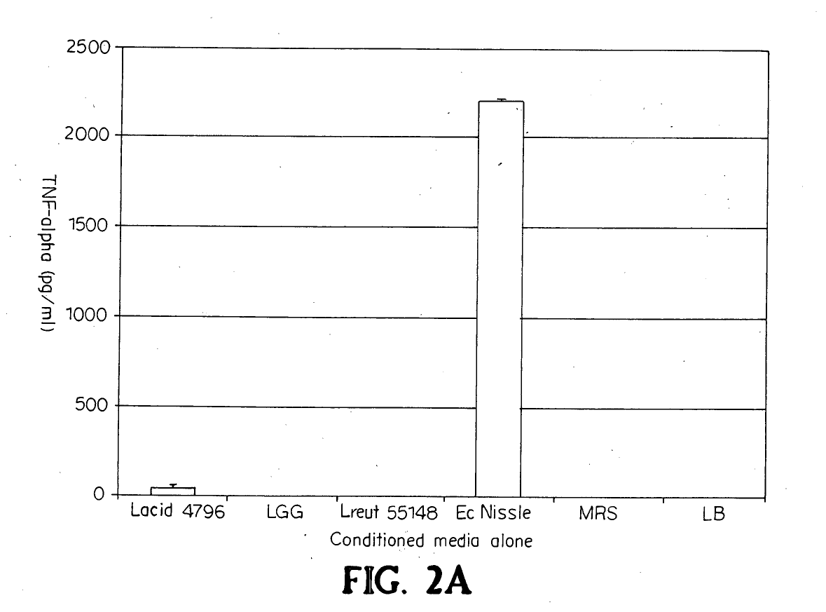

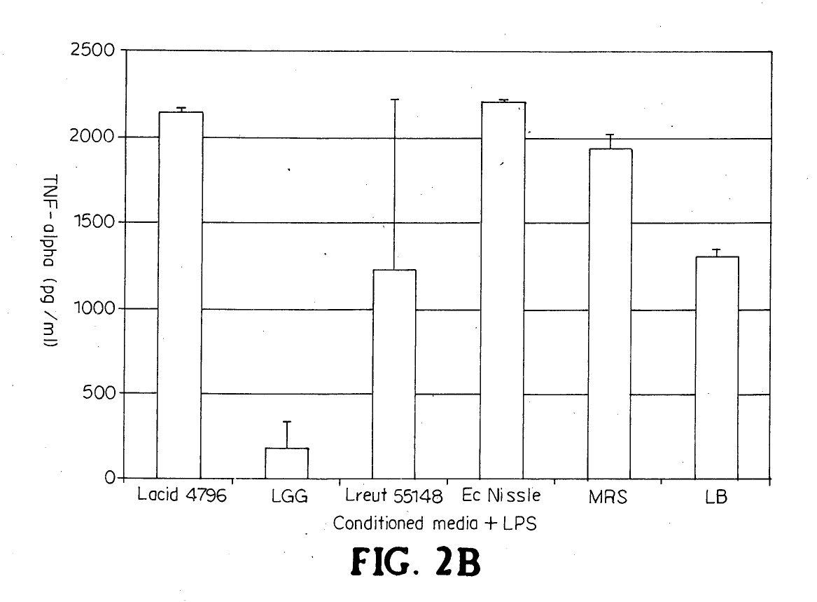

Bacterial cell-free conditioned media from E. coli Nissle and different lactobacilli were tested for effects on pro-inflammatory cytokine output (FIG. 2). Immunomodulatory effects were observed with cell-free conditioned media derived from LGG indicating the presence of a soluble immune response modulating molecule, or immunomodulin(s). In the presence of LGG-conditioned media (LGG-cm), LPS-activated macrophages have a significant decrease in TNF-α secretion when compared to macrophages exposed to LPS alone (p<0.025). The ability of LGG to inhibit LPS-induced TNF-α production in macrophages depended on the relative concentrations of LPS and putative bacterial immunomodulins. As the concentration of LPS is increased, the ability of LGG-cm to modulate TNF-α response is diminished (data not shown). Conversely, maintaining the LPS concentration at 2 ng/well and varying the amount of LGG-cm yielded similar results.

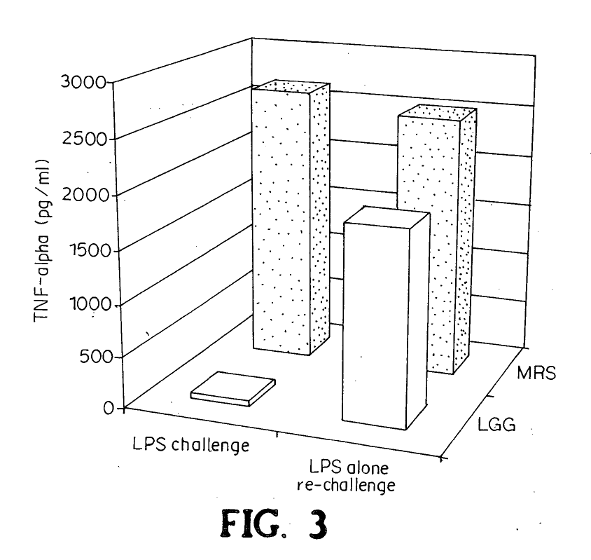

Since the modulatory activity of LGG-cm seemed to be concentration-dependent, we examined whether the ability to inhibit LPS-mediated TNF-α production (with 2 ng LPS/well) by the putative immunomodulin was bacterial-density dependent. LGG-cm collected at 4, 8 and 24 h post-inoculation were compared. These three time points represent early log, mid-log and late logarithmic/early plateau phases of LGG growth, respectively, based on absorbance spectrophotometry. The immunomodulatory activity was most potent in LGG-cm harvested at 24 h, while conditioned media of bacteria in log phase had only partial immunomodulatory activity. Re-challenge experiments were performed to determine the longevity of the TNF-α inhibitory activity. Macrophages were stimulated using LGG-cm with LPS or LPS alone. At the end of 5 h post-activation, cell culture media was removed and replenished with fresh media. After 24 h, both LGG with LPS- or LPS-treated cells were re-challenged with LPS alone. TNF-α was detectable in both groups, showing that the putative immunomodulin blocks TNF-α in a reversible manner (FIG. 3).

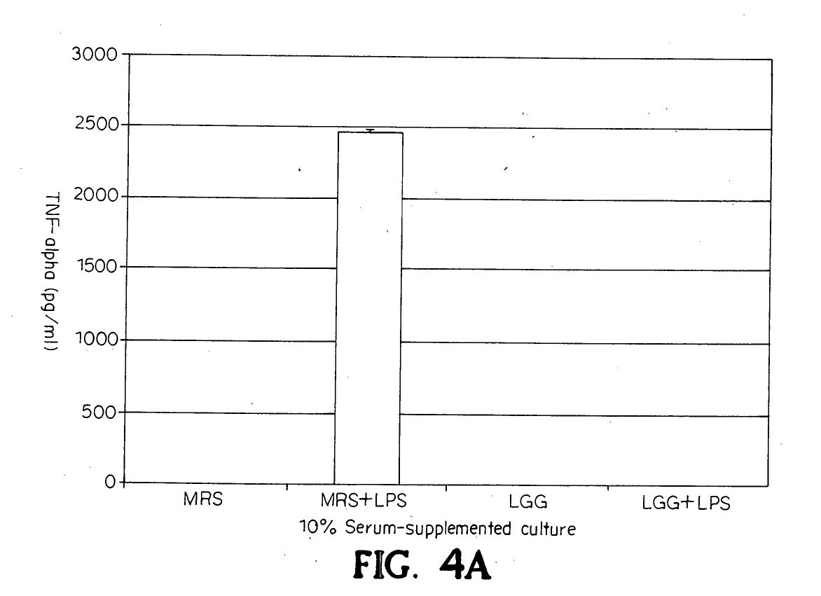

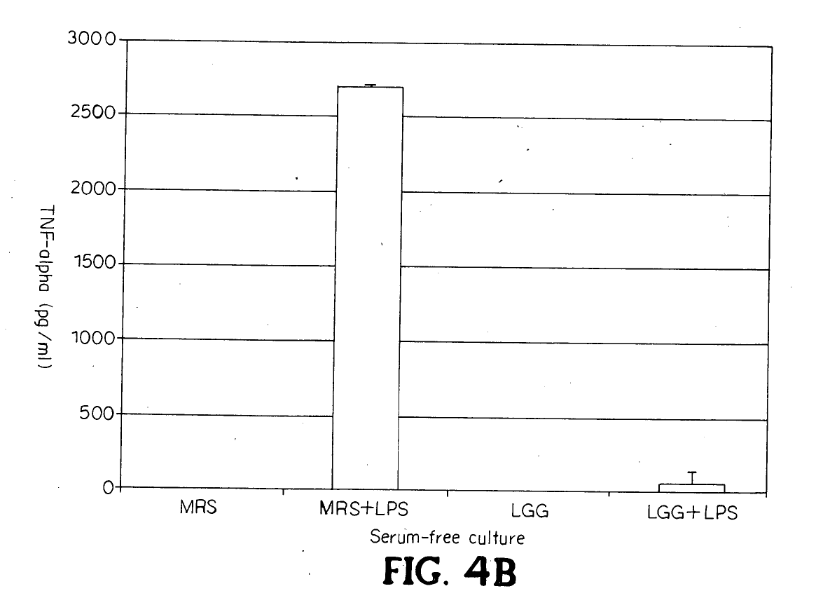

Macrophages and other immune cells recognizing P/CAMPs via PRRs are thought to require soluble co-factors in serum, such as soluble CD14 (sCD14) and LPS-binding proteins (LBP) (Muta and Takeshige, 2001). Bioassays were performed in serum-free media and TNF-α was measured in LPS-exposed cells. In our in vitro system, LPS-induced TNF-α production by macrophages was independent of serum-soluble co-factors, although there was a slight, but insignificant, difference in the production of TNF-α in serum-deprived cells compared to serum-supplemented macrophages. Importantly, LGG immunomodulatory activity was retained in the absence of serum (FIG. 4).

### Example 2

### LGG-Mediated Inhibition of TNF-α Production by LTA-Activated Macrophages

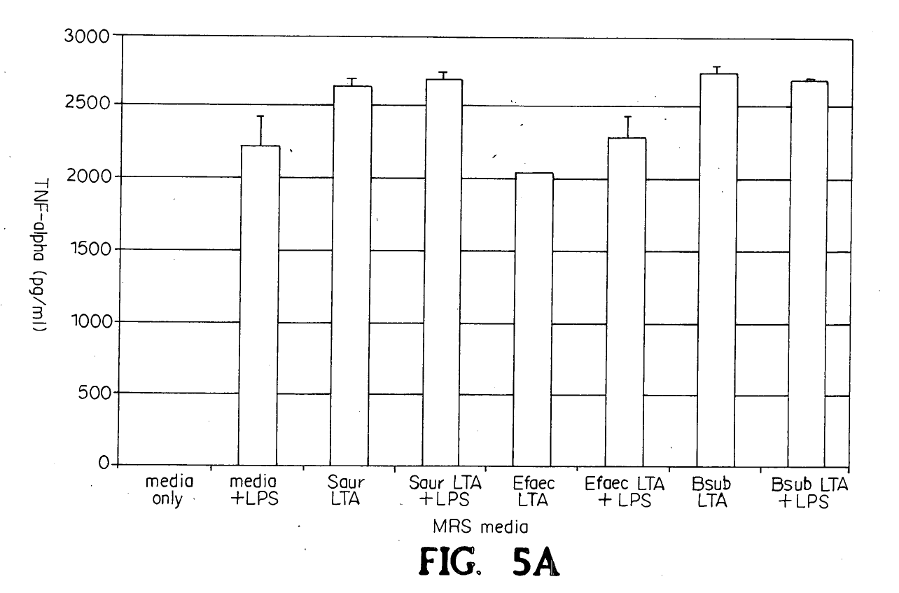

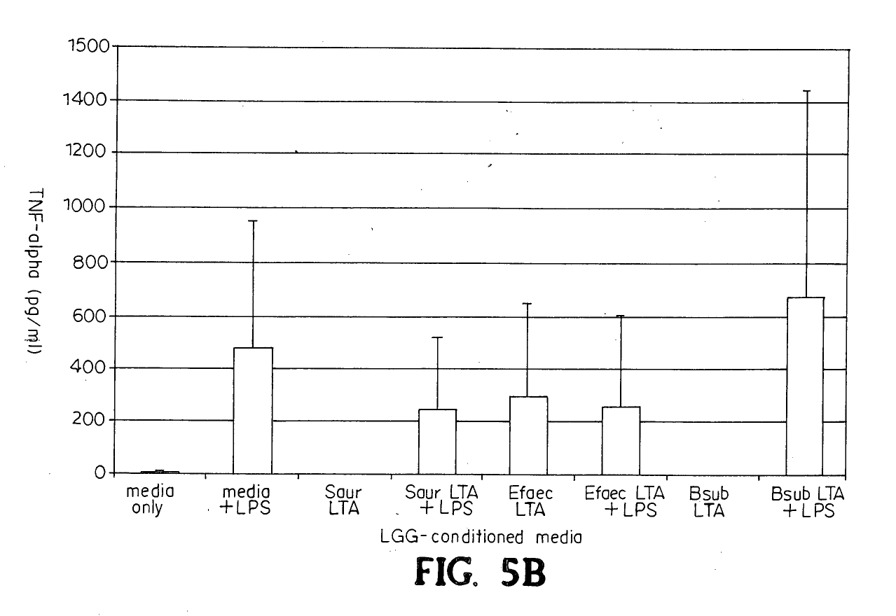

Other pathogen or commensal associated molecular pattern (P/CAMP) biomolecules, such as Gram-positive bacterial lipoteichoic acid (LTA), have been shown to activate macrophages via PRRs (Takeuchi et al., 1999). To explore the Toll-like receptor (TLR2)-mediated pathway, LGG-conditioned media was added to LTA-activated macrophages. Indeed, LGG-cm inhibited TNF-α secretion by macrophages induced by LTA from S. aureus, E. faecalis or B. subtilis. In this assay, LTA was able to induce TNF-α levels that were comparable to that of LPS. It is worth mentioning that while concentrations of LTA used in the bioassays were more than ten times that of LPS (25 ng/50000 cells and 2 ng/50000 cells, respectively), the same amount of LGG-cm inhibited TNF-α secretion for both LTA- and LPS-activated macrophages (see Experimental Procedures). However, when macrophages were exposed to both LPS and LTA, the TNF-α-inhibitory activity of LGG is partially reduced (FIG. 5). These results suggest that dual stimulation of TLR2 and TLR4-mediated pathways partially overcome the block in TNF-α production.

### Example 3

### Evaluation of Cytokine Profiles and Bacterial-Bacterial Antagonism

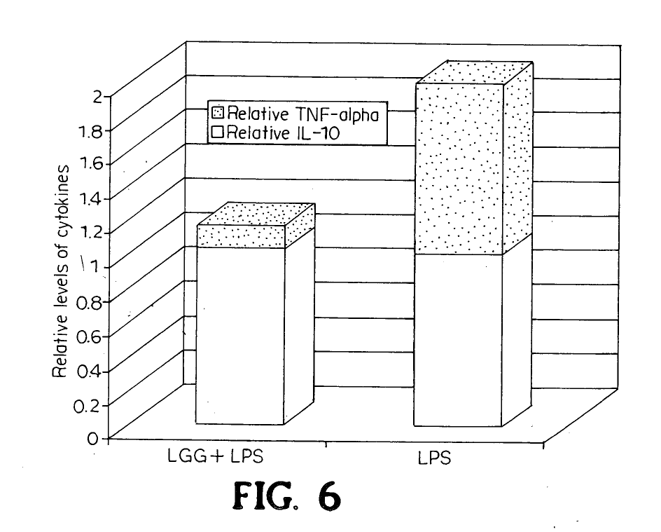

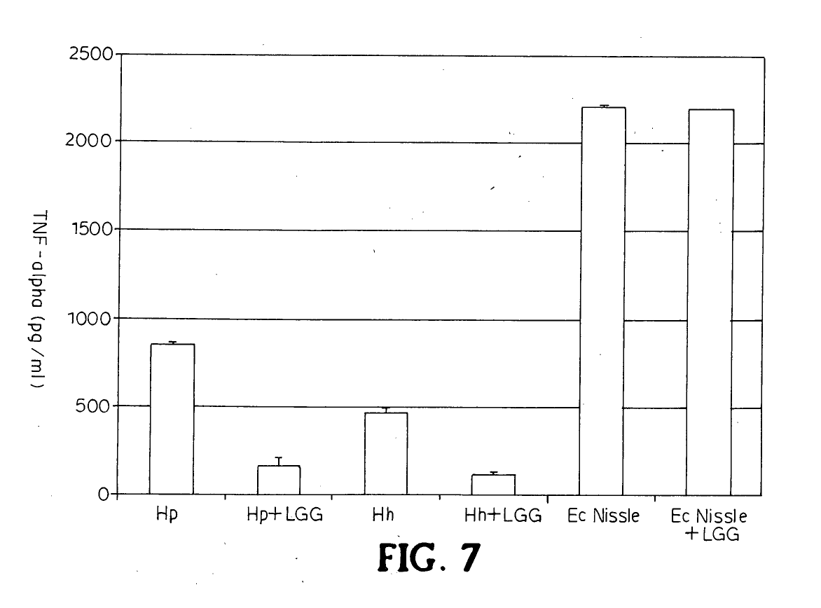

To further understand the implications of TNF-α inhibition by LGG on the cytokine network of the innate immune response, we evaluated cytokine profiles of LPS-stimulated macrophages in the presence or absence of LGG-cm. Bioassays were performed and cytokines quantitated by the Luminex LabMAP 100™ System (Martins et al., 2002). Interleukin-1β (IL-1β), IL-10, IL-12 and TNF-α, but not granulocyte-macrophage colony stimulating factor (GM-CSF), interleukin-6 (IL-6) and interferon-gamma (IFN-γ), were detected in culture supernatants of LPS-stimulated macrophages. Levels of IL-1 β and IL-10 in LGG-treated-LPS-stimulated macrophages were comparable to quantities produced by LPS-stimulated cells. A seven-fold reduction was observed in TNF-α levels in LGG-treated LPS-stimulated cells compared to LPS alone, similar to ELISA data. Interestingly, the levels of IL-10 were unaffected whether macrophages were exposed to LPS alone or co-incubated with LGG-cm. LGG-treated macrophages had diminished TNF-α/IL-10 ratios compared to LPS alone (FIG. 6) indicating a net immunomodulatory effect. Since Gram-negative bacterial-derived products stimulate naive macrophages, we wanted to establish whether LGG could prevent TNF-α production induced by E. coli or pathogenic helicobacters. In our assay, conditioned media of Gram-negative bacteria such as E. coli, H. pylori or H. hepaticus, are capable of inducing TNF-α secretion by macrophages. However, neither H. pylori- or H. hepaticus-derived P/CAMPs present in conditioned media are as potent as E. coli-derived P/CAMPs in stimulating TNF-α secretion in macrophages. Intragenus comparison of macrophage activation shows that H. pylori-conditioned media elicits about 900 ρg/ml TNF-α while H. hepaticus produces approximately half of H. pylori-induced levels. In the presence of LGG-cm, TNF-α induction is significantly inhibited indicating antagonism of LGG-derived immunomodulins versus Helicobacter-derived immunostimulatory factors (p<0.01). It is interesting to note that induction by E. coli is not affected by the addition of LGG-cm. LGG may inhibit TNF-α only when LPS (or an immunostimulatory P/CAMP) of a given nature or particular threshold concentration is present (FIG. 7).

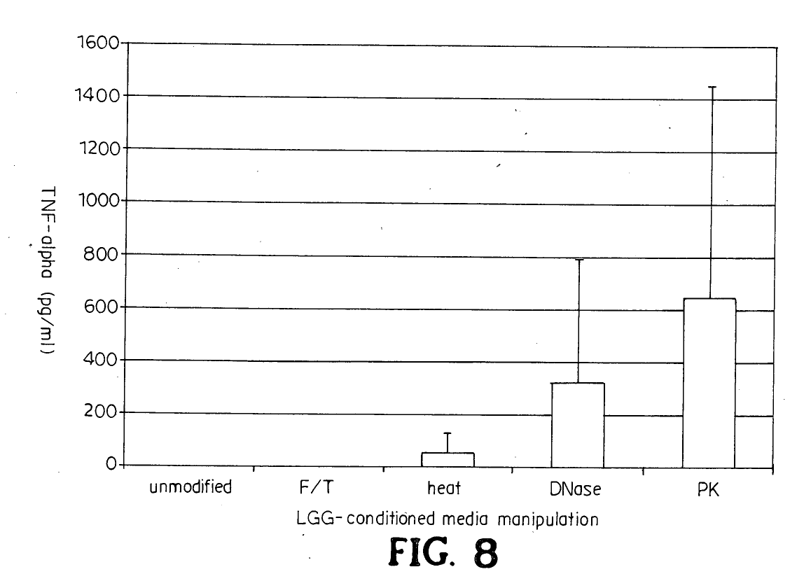

To further characterize this putative immunomodulin, conditioned media from LGG was treated with DNase I, Proteinase K or Protease E. Protease digestion of conditioned media, followed by heat inactivation of proteases, resulted in partial, but significant (p<0.05), loss of TNF-α inhibitory activity of LGG-cm relative to unmodified LGG-cm (FIG. 8). This implies that the putative immunomodulin has a protein or peptide component that inhibits TNF-α production in macrophages.

### Example 4

### Significance of the Present Invention

In summary, these results indicate that L. rhamnosus GG specifically inhibits TNF-α production and reduces TNF-α/IL-10 ratios in a murine macrophage model. The net effect is immunomodulatory in nature. Other Lactobacillus species did not have such a modulatory effect, demonstrating that specific immune effects may be species- or strain-specific. It is believed that in vivo, extracellular pH may influence the immune response (Lardner, 2001). Lactobacillus culture media (MRS broth) is slightly acidic (pH ˜6) and utilization of carbohydrates in the media by lactic acid bacteria further decreases pH to ˜4. The addition of lactobacillus-conditioned media to macrophage cell cultures (RAW Assay) shifted pH to the acidic range and may have impacted TNF-α production. To address the possible impact of pH on TNF-α production, MRS broth was acidified to a pH comparable to lactobacilli conditioned media (approximately pH 4) and used as controls. Acidified MRS did not inhibit LPS-mediated TNF-α production and that acidified MRS alone, could not induce TNF-α production in naive macrophages (data not shown).

Additionally, if lactic acid were to artificially impact TNF-α production, our observation of L. rhamnosus GG-mediated decrease in TNF-α production would be more widespread (i.e. more isolates would exhibit this effect). Most species and isolates of lactobacilli ferment different carbohydrates into lactic acid, when cultured under lowered oxygen tension. Since we have only found TNF-α inhibition in less than ten strains out of over 100 tested, it seems highly unlikely that lactic acid or other acid metabolites impart TNF-α inihibition. Our data is further supported by findings of (Jensen et al, 1990) that lactic acidosis increases TNF-α production in rat peritoneal macrophages.

This effect is serum- and contact-independent, requiring the presence of soluble LGG immunomodulins for complete modulatory activity. Other NF-κB-dependent cytokines such as interleukin-12 (IL-12) are not inhibited and IL-10 production is unaffected. Thus, this modulatory effect appears to be specific for TNF-α and may be NF-κB-independent. Intestinal lactobacilli produce soluble protein factors that presumably bind to cell surface receptors and somehow inhibit synthesis or secretion of TNF-α independent of pro-apoptotic effects or cell necrosis (so that preferably these compounds do not kill human cells and/or damage them by toxic effects).

TNF-α represents a potent pro-inflammatory cytokine produced by activated macrophages which stimulates Th1 immune responses. TNF-α production in LPS-activated macrophages is dependent on NF-κB activation. NF-κB is considered to be a key transcriptional regulator of pro-inflammatory genes important in host innate immune responses. Inhibition of TNF-α production may be secondary to interference with NF-κB activation, blocking transcription of TNF-α. With respect to LGG and murine macrophages, this pathway does not appear to be affected because other NF-κB-regulated genes such as IL-12 are not diminished. Data indicates that TNF-α mRNA levels are unaffected in LPS- or LTA-activated macrophages. Instead, it appears that TNF-α production is specifically inhibited by a post-transcriptional mechanism.

Commensal bacteria are known to produce immunoregulatory factors that may enhance infection in the host by modulating immune responses (Wilson et al., 1998). Such immunomodulins may have important roles in maintaining intestinal health and quenching systemic inflammatory response. Lactobacillus paracasei induces populations of regulatory CD4+ T cells which produce high levels of the modulatory cytokines, IL-10 and transforming growth factor—β (TGF-β) (von der Weid et al., 2001). Lactobacilli modulate cytokine production in bone marrow-derived dendritic cells with a net effect of altering overall cytokine profiles in a species-dependent manner (Christensen et al., 2002). Non-virulent Salmonella strains regulate NF-κB-dependent induction of pro-inflammatory cytokine production by preventing ubiquitination of the NF-κB inhibitory subunit, IκBα (Neish et al., 2000).

Prokaryotes have developed mechanisms for inhibiting pro-inflammatory cytokine responses and facilitating long-term colonization and microbial:host co-existence. Lactobacilli may exert different effects on both mucosal and systemic cytokine levels in rodent models (Ha et al., 1999;Tejada-Simon et al., 1999) and highlight the importance of examining quantitative differences in cytokine synthesis. These seemingly disparate results emphasize the importance of distinguishing experimental studies with lysates versus intact cells or conditioned media. Additionally, different species or strains of any genus may have distinct biologic effects. The biologic unit of importance for pathogenesis and commensalism is ultimately the clone. In support of the strain differences, studies have demonstrated the strain-dependence of immunopotentiating effects of Lactobacillus delbrueckii (Nagafuchi et al., 1999).

Pathogenic bacteria produce proteins that diminish TNF-α expression in host immune cells by different mechanisms and presumably facilitate systemic spread and proliferation. For example, Brucella suis produces a major outer membrane protein, Omp25, that inhibits TNF-α production by human macrophages during infection (Jubier-Maurin et al., 2001). Anthrax lethal factor produced by Bacillus anthracis cleaves two mitogen-activated protein kinases (MAPKKs) in macrophages, causing a substantial reduction in the production of nitrogen oxide (NO) and TNF-α in response to lipopolysaccharide or IFN-γ (Pellizzari et al., 1999). The intestinal pathogen Yersinia enterocolitica expresses a protein YopP that interferes with TNF-α production in murine monocyte-macrophages by interfering with the NF-κB and MAPK pathways (Boland and Cornelis, 1998).

Probiotic Lactobacillus species as well as other probiotic lactic acid bacterial species, have been effective in several animal models and clinical trials. Administration of L. reuteri to IL-10 deficient mice resulted in amelioration of colitis in treated animals and apparent shifts in the nature of the intestinal microbiota (Madsen et al., 1999;Madsen et al., 2000). In the acetic acid-induced rat colitis model, L. reuteri and L. rhamnosus GG yielded beneficial effects and diminished mucosal inflammation (Holma et al., 2001). Different species of Lactobacillus have been included in modern probiotic formulations for the treatment of antibiotic-associated colitis, viral gastroenteritis, and inflammatory bowel disease in human patients. Oral ingestion of Lactobacillus rhamnosus GG has reduced recurrence risk in antibiotic-associated colitis (Bennett et al., 1996). Administration of Lactobacillus reuteri has reduced the length of disease and ameliorated symptoms due to rotaviral gastroenteritis (Shornikova et al., 1997). Finally, the administration of a mixture of Lactobacillus and Bifidobacterium sp. (VSL#3) in ulcerative colitis patients following colectomy has reduced recurrence of flare-ups in chronic pouchitis (Gionchetti et al., 2000).

Probiotic organisms including members of the genus Lactobacillus or other lactic acid bacterial species as known the art offer intriguing possibilities as anti-inflammatory biotherapeutic agents. Increased interest in probiotics for the treatment of inflammatory and infectious diseases of the gastrointestinal tract has generated enthusiasm for new therapeutic regimens, but the optimal bacterial strains for these purposes require further investigation. A more complete understanding of the molecular mechanisms of immunomodulation will facilitate the development of next-generation probiotics and will enhance our understanding of host:microbial interactions. Co-evolution of host and commensal organisms serve as a valuable context for framing the scientific questions as we proceed. Clearly commensal bacteria including lactobacilli interact intimately with the host mucosa beyond simple adherence. The production of surface-bound and secreted factors trigger particular eukaryotic signaling pathways and ultimately affect the production of specific host proteins. Such molecular interactions will shed insights and uncover new mechanisms into the regulation of mucosal inflammation and host immune responses.

### Example 5

### Exemplary Experimental Procedures

Although the following materials and methods are exemplary regarding the present invention, in a specific embodiment they are useful for experiments described in Examples 1-4.

Bacteriologic Methods

Lactobacillus spp. (L. acidophilus ATCC 4796, L. animalis ATCC 35046, L. rhamnosus GG ATCC 53103, L. johnsonii ATCC 33200, L. murinus ATCC 35020, L. plantarum ATCC 14917, L. plantarum ATCC 49445, L. reuteri ATCC 53608, L. reuteri ATCC 55148, L. salivarius ATCC 11471) and E. coli Nissle (obtained from V. Fussing, Statens Serum Institut, Copenhagen , Denmark) were grown in de Man, Rogosa, Sharpe (MRS) and Luria-Bertani (LB) media (Difco, Sparks, Md.), respectively. Overnight cultures of lactobacilli were diluted to an OD₆₀₀ of 1.0 (representing approximately 10⁹ cells/ml) and further diluted 1:10 and grown for an additional 4, 8 and 24 h. Helicobacter pylori Sydney and Helicobacter hepaticus 3B1 were cultured for 48 h in Brucella broth (Difco) supplemented with 10% fetal bovine serum (FBS). Cultures were diluted 1:10 and grown for another 24 and 48 h. Bacterial cell-free conditioned media was collected by centrifugation at 8500 rcf for 10 min at 4° C. Conditioned media was separated from cell pellet and filtered through a 0.22 ρm pore filter unit (Millipore, Bedford, Mass.). Intact UV-killed bacteria were prepared by washing lactobacilli in PBS and re-suspending cells to an OD₆₀₀ of 1. Bacterial cells were exposed to 2400 μjoules of UV₂₅₄ₙₘ light in a Stratalinker® UV Crosslinker (Stratagene, La Jolla, Calif.) and plated out on MRS agar to assess viability. Intactness of UV-killed cells was assessed by Gram-stain morphology.

Manipulation of Conditioned Media

Lactobacillus-conditioned media was treated with degradative enzymes and temperature shifts to determine the nature of immunomodulatory molecules possibly secreted by these microorganisms. Conditioned media was subjected to the following: three cycles of freezing and thawing, 15 min heating at 95° C., 15 min DNase I (Ambion, Austin, Tex.) treatment at Tᵣₒₒₘ, or 20 min digestion at 37° C. with Proteinase K or Protease E (Sigma, St. Louis, Mo.), followed by a 10 minute heat inactivation at 95° C. MRS broth was acidified with hydrochloric acid to a p H comparable to lactobacilli conditioned media (approximately pH 4) and used as controls.

Cell Cultures and Bioassays

Mouse monocyte/macrophage cell lines, RAW 264.7 (ATCC TIB-71) and RAW 264.7 gamma NO (−) (ATCC CRL-2278), were used as reporter cells for studying inflammatory response pathways. RAW 264.7 cells were grown in either Dulbecco's Modified Eagle Medium (for wild-type macrophages) or RPMI Medium 1640 (for gamma NO (−) cells) (Gibco-Invitrogen, Carlsbad, Calif.) supplemented with 10% FBS and 2% antibiotic (5000 units/ml Penicillin and 5 mg/ml Streptomycin, Sigma) at 5% CO₂ 37° C. until 80-90% confluent. Approximately 5×10⁴ cells were seeded into 96-well cell culture clusters and allowed to adhere for 2 h prior to LPS activation and addition of conditioned media. Naive RAW 264.7 cells were exposed to cell-free E. coli or Helicobacter conditioned media, purified lipopolysaccharide (LPS) from E. coli serotype 0127:B8, or lipoteichoic acid (LTA) from Staphylococcus aureus, Enterococcus faecalis and Bacillus subtilis (Sigma). Activation media was made by adding 2 ng LPS or 25 ng LTA to 20 μl conditioned media per well. Macrophages were exposed to either 20 or 200 lactobacilli cells/macrophage in intact cell experiments. Macrophages were either pre-incubated or co-incubated with cell-free Lactobacillus conditioned media. Recombinant mIL-10 (R&D Systems, Minneapolis, Minn.) was used as controls for immunoregulation studies. Cell viability was assessed by the Trypan-blue (Invitrogen) exclusion assay.Cytokine Measurements

Production of TNF-α in macrophage cell culture supernatants was measured with a mouse TNF-α specific sandwich enzyme immunoassay (Biosource, Camarillo, Calif.). To study the cytokine milieu of activated macrophage culture in the presence of putative immunoregulators, mouse-specific cytokine antibody-bead kits for Luminex LabMAP 100™ Systems (Biosource) were used to detect and quantify IL-1β, IL-6, IL-10, IL-12 (p70 and p40 specific), TNF-α, IFN-γ, and GM-CSF in culture supernatants in a Luminex 100 instrument (Luminex Corp., Austin, Tex.).

Statistical Analyses

All experiments were performed at least three times (each time in triplicate) and analyzed using Independent Samples T-Test (SPSS for Windows version 11.0.1, SPSS Inc., Chicago, Ill.) at a significance level of p<0.05. Error bars in figures represent standard deviation (SD).

### Example 6

### ADDITIONAL EMBODIMENTS

Gram stains were done of Lactobacillus rhamnosus GG (LGG), 100× hematoxylin-eosin staining was done of LPS-activated RAW 264.7 macrophages, 40×.

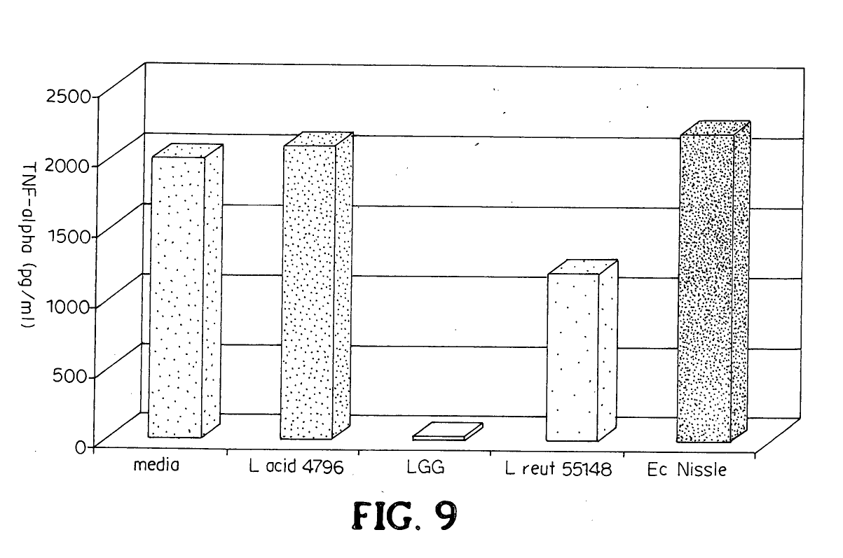

FIG. 9 demonstrates the effect of bacteria-conditioned media on LPS-activated macrophages. Macrophages were activated with a mixture of LPS and bacteria-conditioned media. Culture media was tested 5 h post-activation for TNF-α. L. acidophilus 4796 significantly increased TNF-α production compared to macrophages activated with MRS+LPS only (p<0.01) while L. reuteri ATCC 55148 had no effect. LGG significantly decreased TNF-α production (p<0.01). Gram-negative bacteria such as E. coli, significantly increased TNF-α production compared to culture media alone.

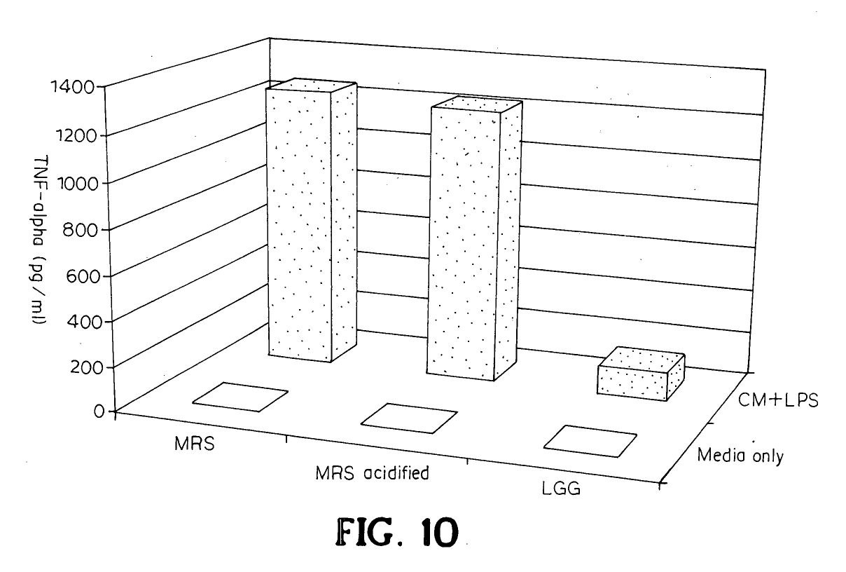

FIG. 10 demonstrates that immunomodulation is not due to pH effects. To control for lactic acid production and reduced pH effects, acidified MRS media (pH 4) was tested and did not affect TNF-α levels without the presence of LGG-cm. Conditioned media derived from other lactic acid bacteria did not inhibit TNF-α secretion and was inconsistent with general pH effects due to lactic acid production.

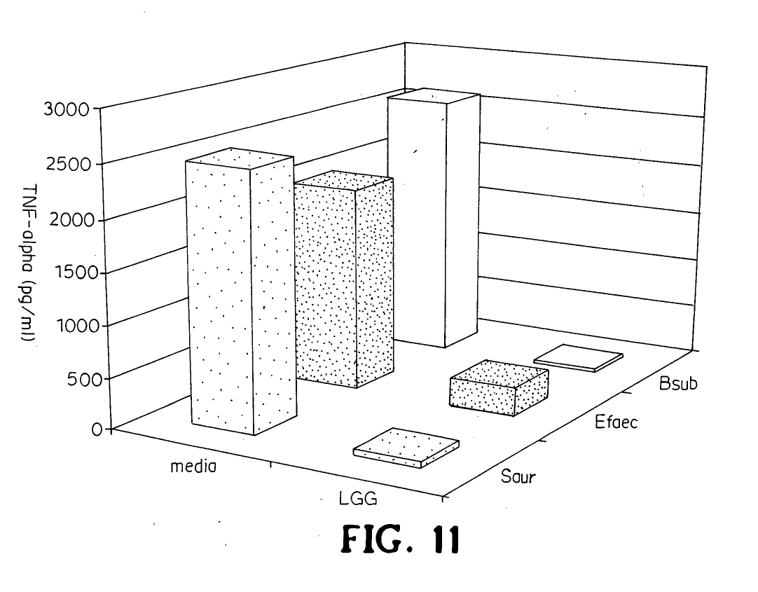

FIG. 11 provides effects of LGG-conditioned media on LTA-activated macrophages. Macrophages were activated with LTA derived from S. aureus, B. subtilis, and E. faecalis. LGG-conditioned media significantly decreased pro-inflammatory cytokine expression in LTA-activated macrophages compared to MRS media alone (p<0.01).

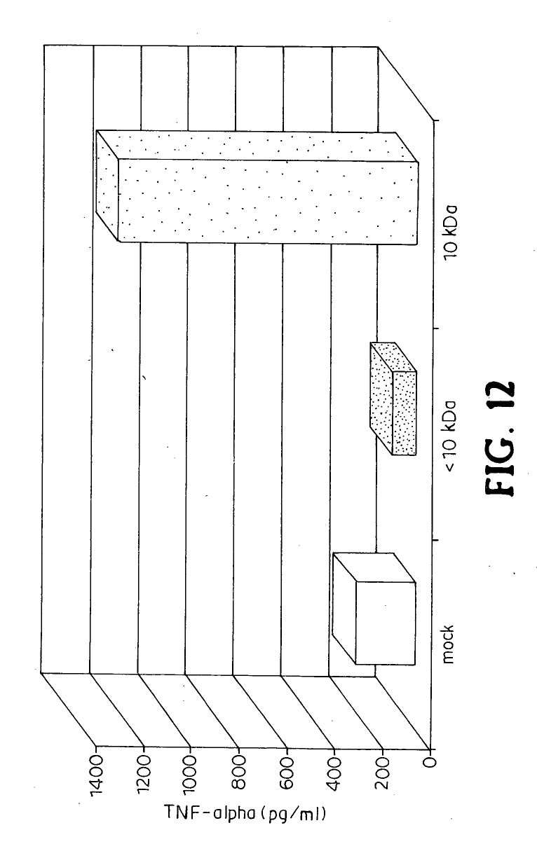

FIG. 12 shows that an immunomodulatory effect is retained in the 10 kDA fraction. LGG-conditioned media was fractionated using size exclusion filters. Inhibition of TNF-α production was observed in the <10 kDa fraction. In contrast, the >10 kDa fraction lost immunomodulatory activity. Taken together with previous data from the inventors, this indicates that a small peptide is responsible for immunomodulation and does not require serum.

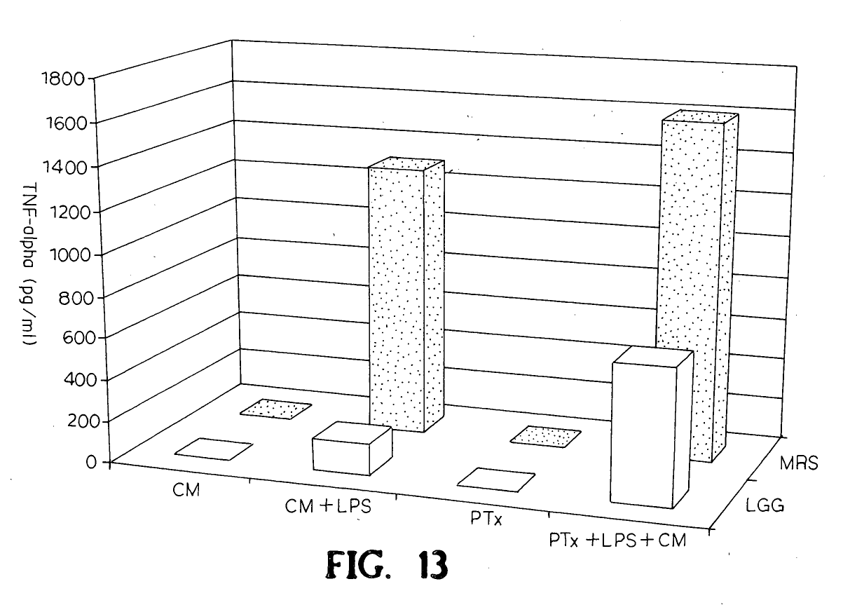

FIG. 13 shows that immunomodulation utilizes heterotrimeric G proteins. Following PTx treatment, RAW 264.7 cells were stimulated with LPS alone or co-cultured with Lactobacillus-conditioned media (CM). The ability of Lactobacillus-conditioned media to exert TNF-inhibitory effects was partially diminished when RAW 264.7 cells were intoxicated with PTx.

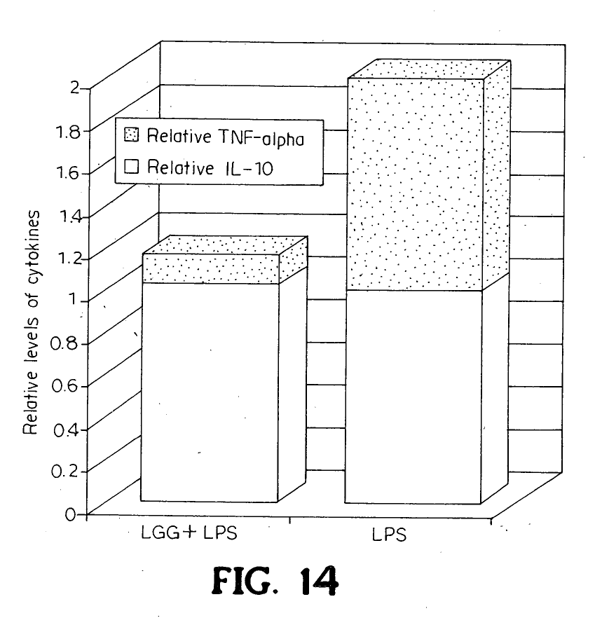

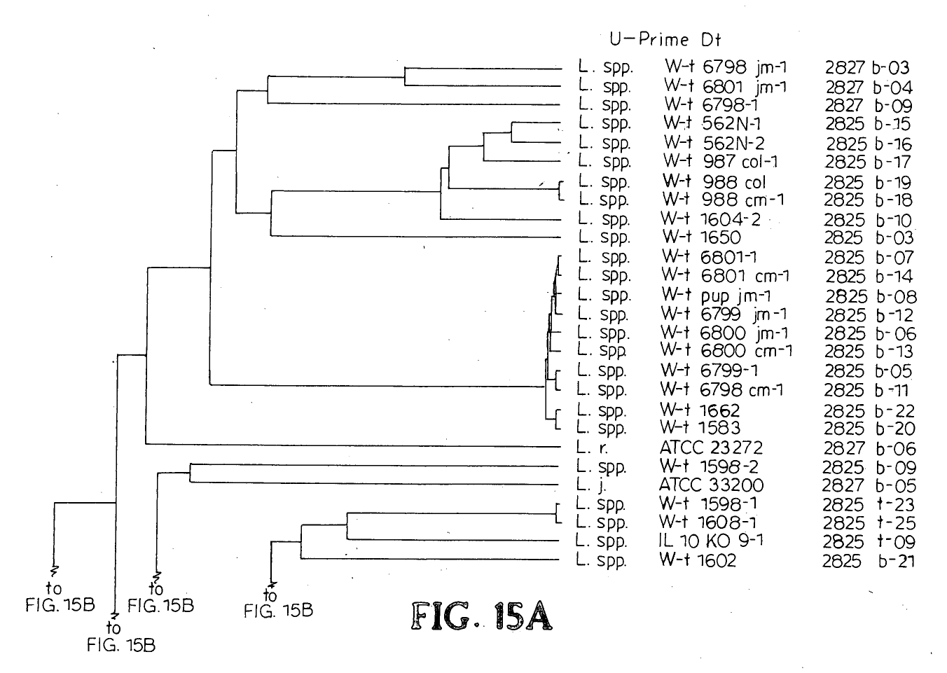

FIG. 14 demonstrates that TNF-α/IL-10 ratios are diminished in presence of LGG. Cytokine levels of LPS-activated macrophages were measured using mouse-specific multi-cytokine antibody-bead sandwich immunoassays in a Luminex 100 instrument. Levels of IL-10 and TNF-α in LGG-cm+LPS-stimulated macrophage were compared relative to macrophages exposed to LPS alone. LGG (L. rhamnosus-conditioned media) and LPS (E. coli O127:B8-derived lipopolysaccharide).

Not shown in the figures herein are similar results obtained with Bifidobacterium and Streptococcus thermophilus.

### Example 7

### Additional Exemplary Procedures

Although the following materials and methods are exemplary regarding the present invention, in a specific embodiment they are useful for experiments described in Example 6.

Bacterial Cultures

L. rhamnosus GG and E. coli Nissle were grown in MRS media and LB media, respectively. Overnight cultures were diluted 1:10 and grown for another 4, 8 and 24 h. Helicobacter pylon Sydney and Helicobacter hepaticus 3B1 were cultured for 48 h in Brucella broth supplemented with fetal bovine serum (FBS). Cultures were diluted 1:10 and grown for another 24 and 48 h. Media conditioned by bacteria was collected by centrifuging cultures.

RAW Bioassay

Peritoneal macrophages from 129 SvEv mice and the monocyte/macrophage cell line, RAW 264.7 gamma NO(−), were used as reporter cells for studying the inflammatory response pathway. Naïve RAW 264.7 gamma NO(−) cells were exposed to cell-free E. coli or Helicobacter conditioned media and purified lipopolysaccharide (LPS) from E. coli serotype 0127:B8 (Sigma, St. Louis, Mo.) or Gram-positive lipoteichoic acid from Staphylococcus aureus, Bacillus subtilis and Enterococcus faecalis (Sigma) while primary macrophages were exposed to LPS or LTA. Macrophages were either pre-incubated or co-incubated with cell-free Lactobacillus conditioned media. For toxin assays, RAW 264.7 macrophages were exposed to a Gi protein inhibitor, pertussis toxin (PTx), in order to ablate Gi protein-dependent responses. Following PTx treatment, RAW 264.7 cells were stimulated with LPS alone or co-cultured with Lactobacillus-conditioned media (CM). Production of TNF-α in cell culture supernatant was measured with a sandwich enzyme immunoassay, Mouse TNF-α ELISA (BioSource, Camarillo, Calif.).

Cytokine Measurements

To study the cytokine milieu of activated macrophage culture in the presence of putative immunoregulators, a mouse multiplex Cytokine Detection System 2 (BioSource) will be used to detect and quantify IL-1 α, IL-2, IL-4, IL-5, IL-6, IL-10, IL-12(p70), TNF-α, IFN-α, and GM-CSF in culture supernatant in a Luminex 100 (Luminex Corp., Austin, Tex.) instrument.

### Example 8

### Intestinal Lactobacillus from Health and IL-10 Deficient Mice

Lactobacillus species have been used as probiotic agents for the treatment of gastrointestinal infections and inflammatory bowel disease. The murine gastrointestinal tract, similar to other mammals including humans, contains sufficient numbers of commensal lactobacilli that are considered important for the maintenance of intestinal health. Interleukin-10 (IL-10) deficient mice develop colitis when colonized with intestinal bacteria due to the absence of an important immunoregulatory cytokine (IL-10).

In order to compare the Lactobacillus microbiota from healthy and IL-10-deficient animals, intestinal lactobacilli were isolated from different regions of the intestine and feces. Candidate murine intestinal lactobacilli were cultured on selective media and screened by Gram stain morphology and selected biochemical tests. Lactobacillus isolates were characterized by detailed biochemical studies, 16S rDNA sequencing, and rep-PCR-based DNA fingerprinting.

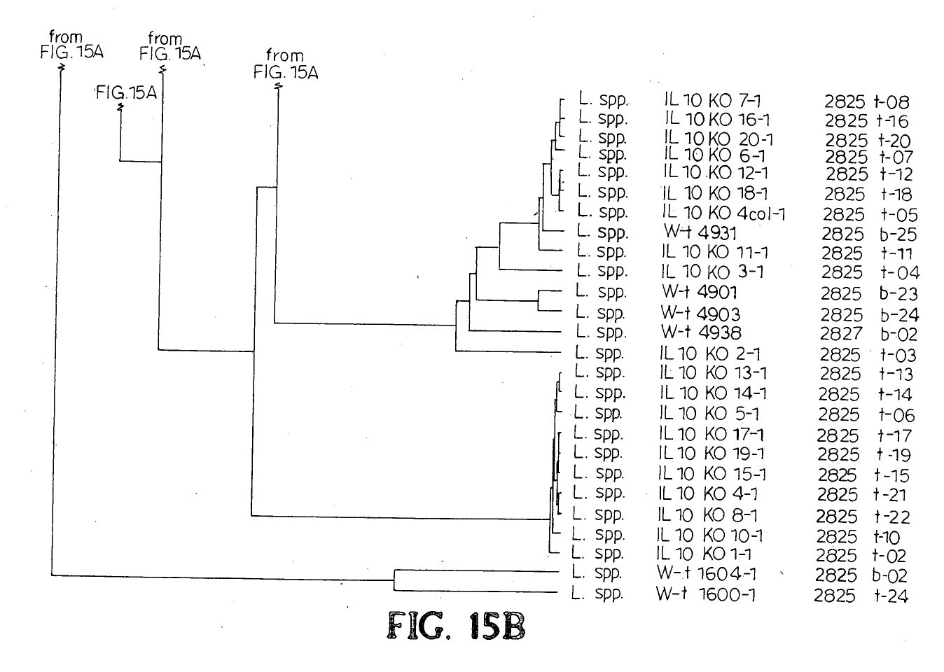

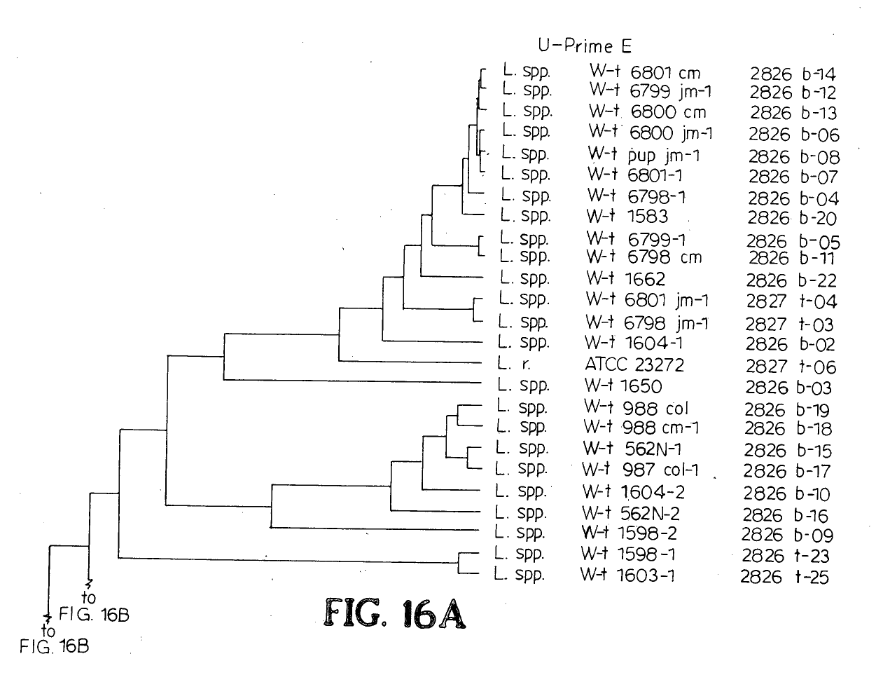

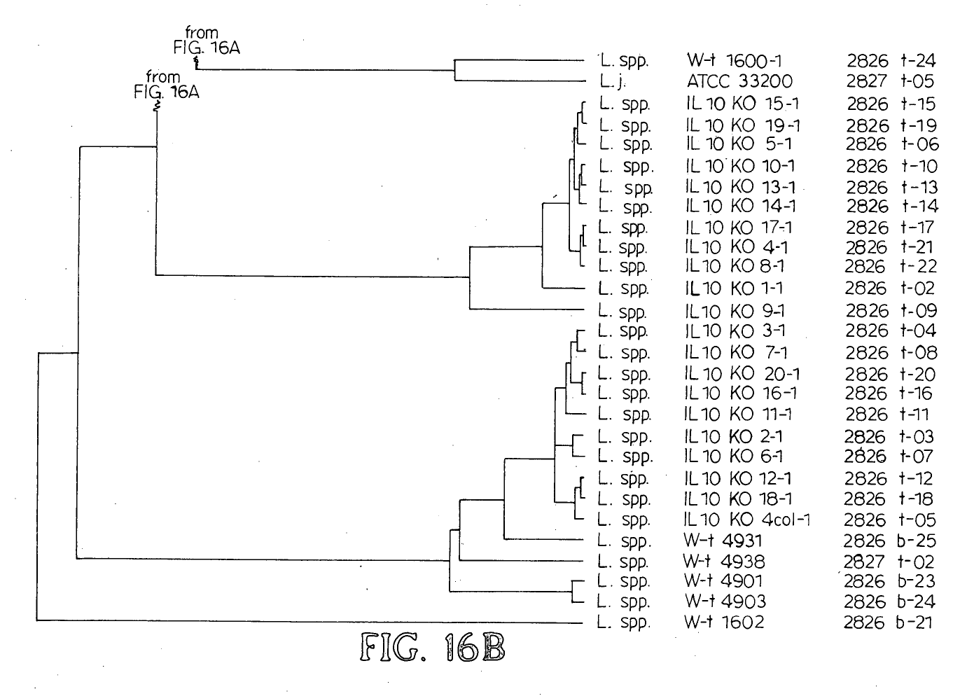

Detailed biochemical and molecular studies of intestinal Lactobacillus isolates highlighted the presence of distinct Lactobacillus populations in healthy versus IL-10-deficient mice. Intestinal Lactobacillus isolates from the same animals and cultured from different regions of the intestinal tract were identical by DNA fingerprinting. Isolates from healthy animals were identified as Lactobacillus reuteri (or L. reuteri/fermentum complex) by biochemical analyses and DNA sequencing. In contrast, isolates from IL-10-deficient mice were identified as Lactobacillus gasseri or Lactobacillus acidophilus by biochemical studies and DNA sequencing. Consistent with these data, isolates from healthy and diseased animals were clearly distinguished by cluster analyses based on rep-PCR-based DNA fingerprinting (FIGS. 15 and 16).

Distinct intestinal Lactobacillus species predominate in healthy animals and IL-10 deficient mice with colitis. The nature of the Lactobacillus microbiota may partly contribute to intestinal health or inflammation and may be relevant for probiotic treatment strategies.

### Example 9

### TNF-α-Inhibitory (“Immunomodulin”) Activity Requires the Presence of G Protein GIα2

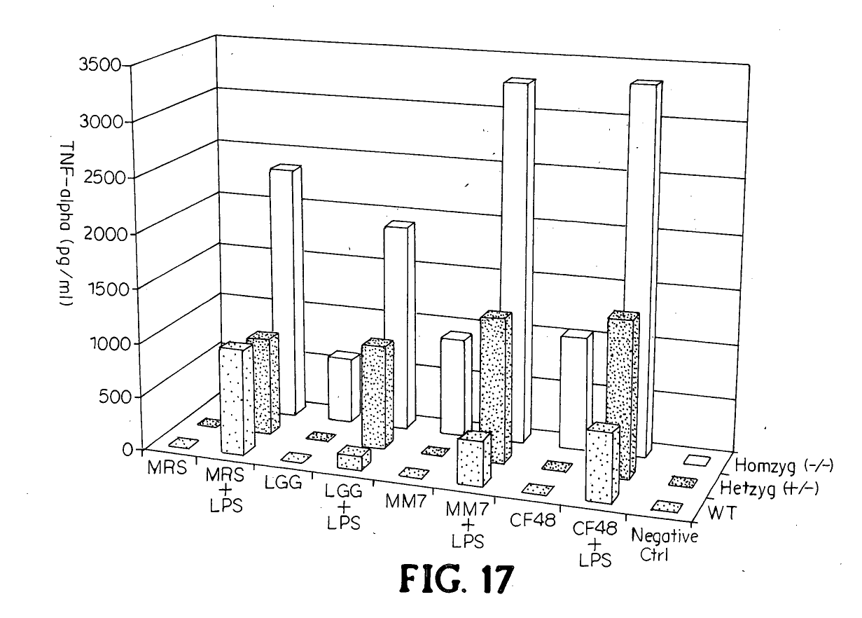

Macrophages from wild type and heterozygous knockout animals secreted comparable levels of TNF-α when stimulated with LPS alone. Heterozygous Giα2^{±} macrophages produced intermediate levels of TNF-α in the presence of Lactobacillus-CM (FIG. 17). Homozygous Giα2-deficient macrophages produced excessive amounts of TNF-α, despite the presence of Lactobacillus-derived CM (FIG. 17). CM derived from Lactobacillus spp. inhibited LPS-induced TNF-α secretion by wild type macrophages, but this effect was abrogated in Giα2-deficient cells (FIG. 17). These results indicate the importance of G protein Giα2 in the modulation of macrophage cytokine responses by commensal Lactobacillus spp. and justify proposed studies of Giα2-deficient mouse models.

### References

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### Patents and Patent Applications

U.S. Pat. No. 4,314,995

U.S. Pat. No. 4,839,281

U.S. Pat. No. 5,032,399

U.S. Pat. No. 6,132,710

U.S. patent application No. 20020019043 A1

### Publications

Ahrne, S., Nobaek, S., Jeppsson, B., Adlerberth, I., Wold, A. E., and Molin, G. (1998b) The normal Lactobacillus flora of healthy human rectal and oral mucosa. J Appl Microbiol 85: 88-94.

Ahrne, S., Nobaek, S., Jeppsson, B., Adlerberth, I., Wold, A. E., and Molin, G. (1998a) The normal Lactobacillus flora of healthy human rectal and oral mucosa. J Appl Microbiol 85: 88-94.

Alander, M., Korpela, R., Saxelin, M., Vilpponen-Salmela, T., Mattila-Sandholm, T., and von Wright, A. (1997) Recovery of Lactobacillus rhamnosus GG from human colonic biopsies. Lett Appl Microbiol 24: 361-364.

Alander, M., Satokari, R., Korpela, R., Saxelin, M., Vilpponen-Salmela, T., Mattila-Sandholm, T., and von Wright, A. (1999) Persistence of colonization of human colonic mucosa by a probiotic strain, Lactobacillus rhamnosus GG, after oral consumption. Appl Environ Microbiol 65: 351-354.

Alvarez-Olmos, M. I. and Oberhelman, R. A. (6-1-2001) Probiotic agents and infectious diseases: a modern perspective on a traditional therapy. Clin Infect Dis 32: 1567-1576.

Aranda, R., B. C. Sydora, P. L. McAllister, S. W. Binder, H. Y. Yang, S. R. Targan, and M. Kronenberg. 1997. Analysis of intestinal lymphocytes in mouse colitis mediated by transfer of CD4+, CD45RBhigh T cells to SCID recipients. J. Immunol. 158:3464-3473.

Banasaz, M., Norin, E., Holma, R., and Midtvedt, T. (2002) Increased enterocyte production in gnotobiotic rats mono-associated with Lactobacillus rhamnosus GG. Appl Environ Microbiol 68: 3031-3034.

Bennett, R. G., Gorbach, S. L., Goldin, B. R., Chang, T. W., Laughon, B. E., Greenough III, W. B., and Bartlett, J. G. (1996) Treatment of relapsing Clostridium difficile diarrhea with Lactobacillus GG. Nutrition Today Supplement 31: 35S-38S.

Boland, A. and Cornelis, G. R. (1998) Role of YopP in suppression of tumor necrosis factor alpha release by macrophages during Yersinia infection. Infect Immun 66: 1878-1884.

Chen, H., Lim, C. K., Lee, Y. K., and Chan, Y. N. (2000) Comparative analysis of the genes encoding 23S-5S rRNA intergenic spacer regions of Lactobacillus casei-related strains. Int J Syst Evol Microbiol 50 Pt 2: 471478.

Christensen, H. R., Frokiaer, H., and Pestka, J. J. (1-1-2002) Lactobacilli differentially modulate expression of cytokines and maturation surface markers in murine dendritic cells. J Immunol 168: 171-178.

Cong, Y., S. L. Brandwein, R. P. McCabe, A. Lazenby, E. H. Birkenmeier, J. P. Sundberg, and C. O. Elson. 1998. CD4+ T cells reactive to enteric bacterial antigens in spontaneously colitic C3H/HeJBir mice: increased T helper cell type 1 response and ability to transfer disease. J. Exp. Med. 187:855-864.

Contractor, N. V., H. Bassiri, T. Reya, A. Y. Park, D. C. Baumgart, M. A. Wasik, S. G. Emerson, and S. R. Carding. 1998. Lymphoid hyperplasia, autoimmunity, and compromised intestinal intraepithelial lymphocyte development in colitis-free gnotobiotic IL-2-deficient mice. J. Immunol. 160:385-394.

Dianda, L., A. M. Hanby, N. A. Wright, A. Sebesteny, A. C. Hayday, and M. J. Owen. 1997. T cell receptor-alpha beta-deficient mice fail to develop colitis in the absence of a microbial environment. Am. J. Pathol. 150:91-97.

Dong, M. Y., Chang, T. W., and Gorbach, S. L. (1987) Effects of feeding Lactobacillus GG on lethal irradiation in mice. Diagn Microbiol Infect Dis 7:1-7.

Garcia-Lafuente, A., M. Antolin, F. Guarner, E. Crespo, A. Salas, P. Forcada, M. Laguarda, J. Gavalda, J. A. Baena, J. Vilaseca, and J. R. Malagelada. 1997. Incrimination of anaerobic bacteria in the induction of experimental colitis. Am. J. Physiol. 272:G10-G15

Gionchetti, P., Rizzello, F., Venturi, A., Brigidi, P., Matteuzzi, D., Bazzocchi, G., Poggioli, G., Miglioli, M., and Campieri, M. (2000) Oral bacteriotherapy as maintenance treatment in patients with chronic pouchitis: a double-blind, placebo-controlled trial. Gastroenterology 119: 305-309.

Gorbach, S. L. (2000a) Probiotics and gastrointestinal health. Am J Gastroenterol 95: S2-S4.

Gorbach, S. L., Chang, T. W., and Goldin, B. (12-26-1987) Successful treatment of relapsing Clostridium difficile colitis with Lactobacillus GG. Lancet 2: 1519.

Ha, C. L., Lee, J. H., Zhou, H. R., Ustunol, Z., and Pestka, J. J. (1999) Effects of yogurt ingestion on mucosal and systemic cytokine gene expression in the mouse. J Food Prot 62: 181-188.

Hessle, C., Hanson, L. A., and Wold, A. E. (1999) Lactobacilli from human gastrointestinal mucosa are strong stimulators of IL-12 production. Clin Exp Immunol 116: 276-282.

Holma, R., Salmenpera, P., Lohi, J., Vapaatalo, H., and Korpela, R. (2001) Effects of Lactobacillus rhamnosus GG and Lactobacillus reuteri R2LC on acetic acid-induced colitis in rats. Scand J Gastroenterol 36: 630-635.

Holzapfel, W. H., Haberer, P., Geisen, R., Bjorkroth, J., and Schillinger, U. (2001) Taxonomy and important features of probiotic microorganisms in food and nutrition. Am J Clin Nutr 73: 365S-373S.

Janeway, C. A., Jr. and Medzhitov, R. (2002) Innate immune recognition. Annu Rev Immunol 20: 197-216.

Jubier-Maurin, V., Boigegrain, R. A., Cloeckaert, A., Gross, A., Alvarez-Martinez, M. T., Terraza, A., Liautard, J., Kohler, S., Rouot, B., Domand, J., and Liautard, J. P. (2001) Major outer membrane protein Omp25 of Brucella suis is involved in inhibition of tumor necrosis factor alpha production during infection of human macrophages. Infect Immun 69: 4823-4830.

Kiijavainen, P. V., ElNezami, H. S., Salminen, S. J., Ahokas, J. T., and Wright, P. F. (1999) Effects of orally administered viable Lactobacillus rhamnosus GG and Propionibacterium freudenreichii subsp. shermanii JS on mouse lymphocyte proliferation. Clin Diagn Lab Immunol 6: 799-802.

Kühn, R., J. Löhler, D. Rennick, K. Rajewsky, and W. Müller. 1993. Interleukin-10-deficient mice develop chronic enterocolitis. Cell 75:263-274

Lien, E., Means, T. K., Heine, H., Yoshimura, A., Kusumoto, S., Fukase, K., Fenton, M. J., Oikawa, M., Qureshi, N., Monks, B., Finberg, R. W., Ingalls, R. R., and Golenbock, D. T. (2000) Toll-like receptor 4 imparts ligand-specific recognition of bacterial lipopolysaccharide. J Clin Invest 105: 497-504.

Lilly, D. M. and Stillwell R. H. (1965) Probiotics: growth promoting factors produced by microorganisms. Science 47: 747-748.

Lowenstein, C. J., Alley, E. W., Raval, P., Snowman, A. M., Snyder, S. H., Russell, S. W., and Murphy, W. J. (10-15-1993) Macrophage nitric oxide synthase gene: two upstream regions mediate induction by interferon gamma and lipopolysaccharide. Proc Natl Acad Sci USA 90: 9730-9734.

Madsen, K. L., Doyle, J. S., Jewell, L. D., Tavernini, M. M., and Fedorak, R. N. (1999) Lactobacillus species prevents colitis in interleukin 10 gene-deficient mice. Gastroenterology 116: 1107-1114.

Madsen, K. L., Doyle, J. S., Tavernini, M. M., Jewell, L. D., Rennie, R. P., and Fedorak, R. N. (2000) Antibiotic therapy attenuates colitis in interleukin 10 gene-deficient mice. Gastroenterology 118: 1094-1105.

Marchant, S., Brailsford, S. R., Twomey, A. C., Roberts, G. J., and Beighton, D. (2001) The predominant microflora of nursing caries lesions. Caries Res 35: 397-406.

Martins, T. B., Pasi, B. M., Pickering, J. W., Jaskowski, T. D., Litwin, C. M., and Hill, H. R. (2002) Determination of Cytokine Responses Using a Multiplexed Fluorescent Microsphere Immunoassay. Am J Clin Pathol 118: 346-353.

Miettinen, M., Lehtonen, A., Julkunen, I., and Matikainen, S. (4-1-2000) Lactobacilli and Streptococci activate NF-kappa B and STAT signaling pathways in human macrophages. J Immunol 164: 3733-3740.

Mori, K., Yamazaki, K., Ishiyama, T., Katsumata, M., Kobayashi, K., Kawai, Y., Inoue, N., and Shinano, H. (1997) Comparative sequence analyses of the genes coding for 16S rRNA of Lactobacillus casei-related taxa. Int J Syst Bacteriol 47: 54-57.

Muta, T. and Takeshige, K. (2001) Essential roles of CD14 and lipopolysaccharide-binding protein for activation of toll-like receptor (TLR)2 as well as TLR4 Reconstitution of. Eur J Biochem 268: 45804589.

Nagafuchi, S., Takahashi, T., Yajima, T., Kuwata, T., Hirayama, K., and Itoh, K. (1999) Strain dependency of the immunopotentiating activity of Lactobacillus delbrueckii subsp. bulgaricus. Biosci Biotechnol Biochem 63: 474479.

Neish, A. S., Gewirtz, A. T., Zeng, H., Young, A. N., Hobert, M. E., Karmali, V., Rao, A. S., and Madara, J. L. (9-1-2000) Prokaryotic regulation of epithelial responses by inhibition of IkappaB-alpha ubiquitination. Science 289: 1560-1563.

Onderdonk, A. B., M. L. Franklin, and R. L. Cisneros. 1981. Production of experimental ulcerative colitis in gnotobiotic guinea pigs with simplified microflora. Infect. Immun. 32:225-231.

Pavlova, S. I., Kilic, A. O., Kilic, S. S., So, J. S., Nader-Macias, M. E., Simoes, J. A., and Tao, L. (2002) Genetic diversity of vaginal lactobacilli from women in different countries based on 16S rRNA gene sequences. J Appl Microbiol 92: 451459.

Pellizzari, R., Guidi-Rontani, C., Vitale, G., Mock, M., and Montecucco, C. (11-26-1999) Anthrax lethal factor cleaves MKK3 in macrophages and inhibits the LPS/IFNgamma-induced release of NO and TNFalpha. FEBS Lett 462: 199-204.

Pessi, T., Sutas, Y., Hurme, M., and Isolauri, E. (2000) Interleukin-10 generation in atopic children following oral Lactobacillus rhamnosus GG. Clin Exp Allergy 30: 1804-1808.

Poltorak, A., He, X., Smimova, I., Liu, M. Y., Huffel, C. V., Du, X., Birdwell, D., Alejos, E., Silva, M., Galanos, C., Freudenberg, M., Ricciardi-Castagnoli, P., Layton, B., and Beutler, B. (12-11-1998) Defective LPS signaling in C3H/HeJ and C57BL/10ScCr mice: mutations in Tlr4 gene. Science 282: 2085-2088.

Raschke, W. C., Baird, S., Ralph, P., and Nakoinz, I. (1978) Functional macrophage cell lines transformed by Abelson leukemia virus. Cell 15: 261-267.

Rath H C, Schultz M, Freitag R, Dieleman L A, Li F, Linde H J, Scholmerich J, Sartor R B. Different subsets of enteric bacteria induce and perpetuate experimental colitis in rats and mice. Infect Immun. April 2001; 69(4):2277-85.

Reuter, G. (2001) The Lactobacillus and Bifidobacterium microflora of the human intestine: composition and succession. Curr Issues Intest Microbiol 2: 43-53.

Schwandner, R., et al. (1999) Peptidoglycan- and lipoteichoic acid-induced cell activation is mediated by toll-like receptor 2. J Biol Chem 274: 17406-17409.

Shornikova, A. V., Casas, I. A., Mykkanen, H., Salo, E., and Vesikari, T. (1997) Bacteriotherapy with Lactobacillus reuteri in rotavirus gastroenteritis. Pediatr Infect Dis J 16: 1103-1107.

Takeuchi, O., et al. (1999) Differential roles of TLR2 and TLR4 in recognition of gram-negative and gram-positive bacterial cell wall components. Immunity 11: 443-451.

Tannock,G. W. (1997) Normal microbiota of the gastrointestinal tract of rodents. In Gastrointestinal Microbiology. Mackie,R. I., White,B. A., and Isaacson,R. E. (eds.) New York: Chapman and Hall, pp. 187-215.

Tejada-Simon, M. V., Ustunol, Z., and Pestka, J. J. (1999) Effects of lactic acid bacteria ingestion of basal cytokine mRNA and immunoglobulin levels in the mouse. J Food Prot 62: 287-291.

Veltkamp, C., S. L. Tonkonogy, Y. P. de Yong, W. B. Grenther, E. Balish, C. Terhorst, and R. B. Sartor. 2001. Continuous stimulation by normal luminal bacteria is essential for the development and perpetuation of colitis in Tg26 mice after bone marrow transplantation or adoptive transfer. Gastroenterology 120:900-913.

Von der Weid T., Bulliard, C., and Schiffrin, E. J. (2001) Induction by a lactic acid bacterium of a population of CD4(+) T cells with low proliferative capacity that produce transforming growth factor beta and interleukin-10. Clin Diagn Lab Immunol 8: 695-701.

Wilson, M., Seymour, R., and Henderson, B. (1998) Bacterial perturbation of cytokine networks. Infect Immun 66: 2401-2409.

Yamada, T., E. Deitch, R. D. Specian, M. A. Perry, R. B. Sartor, and M. B. Grisham. 1993. Mechanisms of acute and chronic intestinal inflammation induced by indomethacin. Inflammation 17:641-662.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A compound secreted from lactic acid bacteria that comprises anti-inflammation activity.

2. The compound of claim 1, wherein said lactic acid bacteria are selected from the group consisting of Lactobacillus is L. acidophilus, L. animalis, L. rhamnosus GG, L. johnsonii, L. murinus, L. plantarum, L. reuteri, L. salivarius, L. paracasei, L. delbrueckii, L. fermentum, L. brevis, L. buchneri, L. kefi, L. casei L. curvatus, L. coryniformis, Brevibacterium, Streptococcus thermophilus, and a mixture thereof.

3. The compound of claim 1, wherein said compound is a polypeptide.

4. The compound of claim 1, wherein said compound further comprises receptor-binding activity.

5. The compound of claim 1, wherein said compound further comprises cytokine expression regulating activity, chemokine expression regulating activity, or both.

6. A kit comprising the compound of claim 1.

7. A kit that comprises at least one isolated bacterium that produces claim 1.

8. An isolated bacterium that produces the compound of claim 1.

9. The bacterium of claim 8, wherein said bacterium is further defined as being capable of secreting the compound of claim 1.

10. The bacterium of claim 8, wherein said bacterium is Lactobacillus.

11. A culture comprising the bacterium of claim 8.

12. A kit comprising the bacterium of claim 8.

13. A method of reducing cytokine expression in a cell, comprising the step of administering to the cell a compound secreted from lactic acid bacteria.

14. The method of claim 13, wherein said cytokine expression is reduced post-transcriptionally.

15. The method of claim 13, wherein said method further comprises binding of said secreted compound to a G protein receptor.

16. The method of claim 13, wherein said cytokine is TNF-α.

17. The method of claim 13, wherein said cell is an immune cell.

18. The method of claim 17, wherein said immune cell is a macrophage.

19. A method of inhibiting inflammation in an individual, comprising the step of delivering a therapeutically effective amount of lactic acid bacteria to the individual, wherein said lactic acid bacteria inhibit said inflammation by a contact-independent mechanism.

20. The method of claim 19, wherein said lactic acid bacteria are further defined as producing a soluble compound that binds to a receptor on an immune cell.

21. The method of claim 20, wherein the method is further defined as inhibiting, at least partially, in said cell cytokine production, cytokine secretion, chemokine production, or a combination thereof.

22. The method of claim 21, wherein said inhibiting step is further defined as comprising inhibiting said cytokine production, cytokine secretion, chemokine production, or a combination thereof, through inhibitory heterotrimeric G (Gi) protein activity.

23. The method of claim 21, wherein said cytokine is TNF-α.

24. The method of claim 21, wherein said chemokine is IL-8.

25. The method of claim 19, wherein the lactic acid bacteria are administered in combination with at least one additional therapeutic agent.

26. The method of claim 25, wherein the at least one therapeutic agent is selected from the group consisting of corticosteroids, sulphasalazine, derivatives of sulphasalazine, immunosuppressive drugs, cyclosporin A, mercaptopurine, azathioprine, and a mixture thereof.

27. The method of claim 19, wherein said individual is stricken with colitis, arthritis, synovitis, polymyalgia rheumatica, myositis, or sepsis.

28. Lactic acid bacteria secretions, said secretions being polypeptides, and said secretions having anti-inflammatory activity.
